(19) United States
(12) Patent Application Publication
(10) Pub. No.: US 2025/0179034 A1
(43) Pub. Date: Jun. 5, 2025

(54) IONIZABLE LIPIDS AND COMPOSITIONS COMPRISING SAME

(71) Applicant: BARCODE NANOTECH LTD., Nazareth (IL)

(72) Inventors: Ronen EAVRI, Binyamina (IL); Annie SABBAH, Haifa (IL); Felix BADINTER, Karmiel (IL); Aaron ROSENBLOOM, Karmiel (IL); Or ROZEN, Afula (IL)

(21) Appl. No.: 18/843,279

(22) PCT Filed: Mar. 2, 2023
(86) PCT No.: PCT/IL2023/050216

§ 371 Date: Sep. 2, 2024

Related U.S. Application Data
(60) Provisional application No. 63/315,759, filed on Mar. 2, 2022.

(51) Int. Cl. C07D 257/02; A61K 9/51; A61K 48/00; C07C 323/60; C07D 233/64; C07D 473/34; C07F 9/09; C12N 15/88

(52) U.S. Cl. CPC C07D 257/02; A61K 9/5123; A61K 48/0041; C07C 323/60; C07D 233/64; C07D 473/34; C07F 9/091; C12N 15/88

(57) ABSTRACT

One or more ionizable lipid(s) and lipid nanoparticles comprising same are provided. Pharmaceutical compositions comprising the lipid nanoparticles encapsulating an active agent are also provided.

### CROSS-REFERENCE TO RELATED APPLICATIONS

This application claims the benefit of priority of U.S. Provisional Patent Application No. 63/315,759, titled “IONIZABLE LIPIDS AND COMPOSITIONS COMPRISING SAME”, filed Mar. 2, 2022, the contents of which are incorporated herein by reference in their entirety.

### FIELD OF INVENTION

The present invention is directed to ionizable lipids and lipid nanoparticles comprising same and use thereof in diagnostic, therapeutic and theranostic compositions.

### BACKGROUND OF THE INVENTION

New delivery methods for therapeutic and diagnostic compounds are in constant development. Although lipid-based nanoparticles are a well-known delivery modality, these agents are also constantly undergoing improvement. Among other concerns, the ability of a therapeutic carrier to effectively load the active agent, as well as any potential accessory agents such as nucleic acid molecules, is important for accurate dosing, decreasing drug loss/cost and streamlining methods of drug production.

In particular lipids that are positively charged at low pH are highly useful for loading nucleic acids which have a net negative charge. However, positively charged lipids have been found to be toxic when administered systemically and also show poor biodistribution that they tend to adhere to cells and tissues. One solution to this problem is ionizable lipids, which are lipids that are charged at one pH and neutral at another. In particular, ionizable lipids that are positive at low pH (allowing for efficient loading) and neutral at physiological pH (lessening toxicity and improving biodistribution) are highly sought.

Although, several such ionizable lipids are known there is a constant need for new and superior ionizable lipids. The provision of new and superior ionizable lipids will greatly enhance the drug delivery arena and provide new compositions and modalities for delivery of active agent, therapeutics and diagnostic agents to subject in general and specific locations in the body in particular.

### SUMMARY OF THE INVENTION

The present invention provides new compounds comprising an ionizable moiety. In particular, new ionizable lipids and nanoparticles comprising same are provided. Compositions comprising the nanoparticles, which are useful for therapeutic, diagnostic and theranostic methods are also provided.

According to a first aspect, there is provided a compound represented by Formula 1:

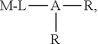

wherein:
M represents an ionizable moiety; the ionizable moiety is selected from (i) an ionizable moiety comprising a heteroaryl; (ii) an ionizable moiety having a binding affinity to a CNS receptor; and (iii) an ionizable moiety comprising a squaramide; L represents a spacer or is a bond; A represents a N, O, S, or C(H)₁₋₂ as allowed by valency; each R independently represents H, a hydrocarbon comprising between 10 and 50 carbon atoms, or is absent, and at least one R is the hydrocarbon; the hydrocarbon comprises a C20-C50 alkyl, a C20-C50 alkenyl, a C20-C50 alkyl or alkenyl comprising one or more heteroatoms, or the hydrocarbon is represented by Formula I:

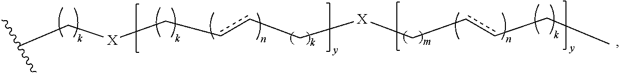

wherein:
k, and y, are integers each independently being between 1 and 10; n and m are integers each independently being between 0 and 10; each X independently comprises (i) CH₂, CHR₁, NR₁, NH, O, S, or phosphate; or (ii)

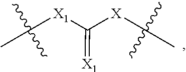

wherein each X₁ is independently O, NH, or S, and wherein X is CH₂, CHR₁, NR₁, NH, O, or S; and wherein R₁ comprises a C1-C20 alkyl, a C1-C20 alkenyl, a C20-C50 alkyl or alkenyl comprising one or more heteroatoms, or the hydrocarbon of Formula I.

According to some embodiments, the heteroaryl of the ionizable moiety is characterized by a pKa between 6 and 7.

According to some embodiments, the heteroaryl is imidazole.

According to some embodiments, the targeting moiety is selected from the group consisting of adenine, adenosine, glutathione, methylxanthine, caffeine, or any combination thereof.

According to some embodiments, the ionizable moiety has a molecular weight (MW) of less than 1000 Da.

According to another aspect, there is provided a compound represented by Formula 2:

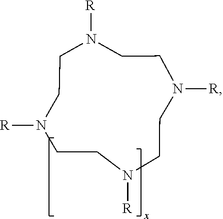

### or by Formula 3:

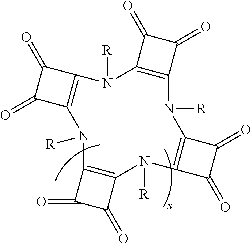

wherein: x is between 1 and 4; each R independently represents H, a hydrocarbon comprising between 10 and 50 carbon atoms, or is absent, and at least one R is the hydrocarbon; the hydrocarbon comprises a C20-C50 alkyl, a C20-C50 alkenyl, a C20-C50 alkyl or alkenyl comprising one or more heteroatoms, or the hydrocarbon is represented by Formula I:

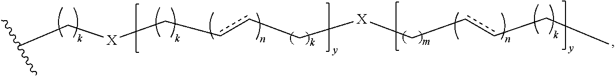

wherein: k, and y, are integers each independently being between 1 and 10; n and m are integers each independently being between 0 and 10; each X independently comprises (i) CH₂, CHR₁, NR₁, NH, O, S, or phosphate;

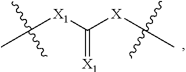

or (ii) wherein each X₁ is independently O, NH, or S, and wherein X is CH₂, CHR₁, NR₁, NH, O, or S; and wherein R¹ comprises a C1-C20 alkyl, a C1-C20 alkenyl, a C20-C50 alkyl or alkenyl comprising one or more heteroatoms, or the hydrocarbon of Formula I.

In one embodiment, the compound of the invention is selected from:

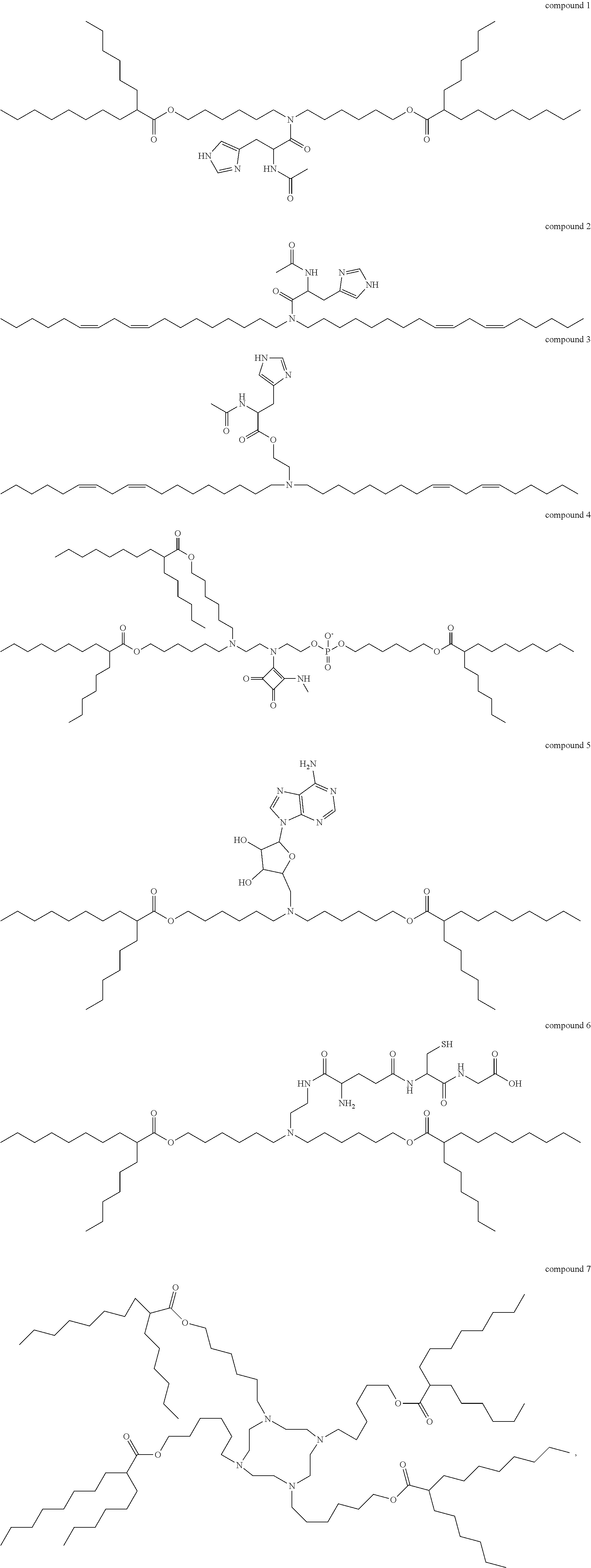

and wherein the compound includes any salt, any stereoisomer, or any tautomer thereof.

According to another aspect, there is provided a nanoparticle comprising a core and a shell:
(i) the shell comprises a lipid, and at least one compound of the invention;
(ii) the core is an aqueous core, comprising an active agent;
and wherein an average size of the nanoparticle is in a range between 30 and 300 nm.

According to some embodiments, a molar concentration of the compound within the nanoparticle is between 10 and 80 mol %.

According to some embodiments, the shell further comprises a sterol.

According to some embodiments, a molar concentration of the sterol within the nanoparticle is between 20 and 60 mol %.

According to some embodiments, a molar concentration of the lipid within the nanoparticle is between 5 and 40 mol %.

According to some embodiments, the lipid is a liposome forming lipid.

According to some embodiments, the lipid further comprises a PEG-lipid.

According to some embodiments, the nanoparticle is a lipid nanoparticle.

In one embodiment, the nanoparticle is a lipid nanoparticle (LNP); and optionally wherein said LNP is characterized by an average particle size between about 50 and about 250 nm, as determined by dynamic light scattering (DLS).

In one embodiment, the LNP comprises a molar concentration of: (i) said compound between 30 and 60%, (ii) the lipid between 5 and 50%, (iii) the sterol between 25 and 50%, and (vi) the PEG-lipid between 0.5 and 5%; and wherein the active agent is a polynucleotide.

In one embodiment, the LNP comprises a molar concentration of (i) the compound about 55%, (ii) the lipid about 10%, (iii) the sterol about 30.5%, and (vi) the PEG-lipid about 4.5%; wherein the compound is compound 5.

In one embodiment, the LNP has affinity to a brain of a subject, and wherein N:P ratio of the LNP is about 3.

In one embodiment, the LNP comprises a molar concentration of (i) the compound about 35%, (ii) the lipid about 25%, (iii) the sterol about 38.5%, and (vi) the PEG-lipid about 1.5%; wherein the compound is compound 1.

In one embodiment, the LNP has affinity to a lung of a subject; and wherein N:P ratio of the LNP is about 9.

In one embodiment, the LNP comprises a molar concentration of (i) the compound about 50%, (ii) the lipid about 10%, (iii) the sterol about 38.5%, and (vi) the PEG-lipid about 1.5-%; wherein the compound is compound 2.

In one embodiment, the LNP has affinity to a heart of a subject; wherein N:P ratio of the LNP is about 4.

In one embodiment, the LNP comprises a molar concentration of (i) the compound about 50%, (ii) the lipid about 10%, (iii) the sterol about 38.5%, and (vi) the PEG-lipid about 1.5%.

In one embodiment, (i) the compound is compound 5; wherein N:P ratio of the LNP is about 4; and wherein the LNP has affinity to a tumor tissue, or (ii) the compound is compound 7; wherein N:P ratio of the LNP is about 4; and wherein the LNP has affinity to a spleen of a subject.

According to another aspect, there is provided a pharmaceutical composition comprising a plurality of nanoparticles of the invention and a pharmaceutically acceptable carrier.

According to some embodiments, the pharmaceutical composition comprises an effective amount of the active agent.

According to some embodiments, the pharmaceutical composition is formulated for systemic administration, administration to a subject, or both.

According to some embodiments, the pharmaceutical composition is for use in a diagnostic, therapeutic or theranostic method comprising administering the pharmaceutical composition to a subject in need thereof.

According to some embodiments, the method is a theranostic method and wherein the nanoparticle comprises an active agent and a nucleic acid molecule uniquely identifying the active agent.

According to some embodiments, the pharmaceutical composition comprises a plurality of types of nanoparticles wherein the plurality comprises at least two nanoparticle types that differ in their lipid composition, their active agent, their nucleic acid molecule or a combination thereof.

According to some embodiments, the nucleic acid molecule uniquely identifies each nanoparticle type.

According to some embodiments, the method comprises administering to a subject a plurality of types of nanoparticles that differ in their lipid composition and are identified by unique nucleic acid molecules and determining the biodistribution of the types of nanoparticles in the subject by the presence of the unique nucleic acid molecules in tissues or cell types of the subject.

According to another aspect, ether is provided a method for delivering of an active agent to a specific tissue of a subject, comprising administering to the subject the pharmaceutical composition of the invention.

In one embodiment, the tissue is selected from tumor, brain tissue, lung tissue, spleen tissue, liver tissue, kidney tissue, and heart tissue.

In one embodiment, the active agent is a polynucleic acid.

In one embodiment, the method comprises administering a therapeutically effective amount of the pharmaceutical composition.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIGS. 1A-1F are bar graphs representing in-vivo accumulation (barcode copies per mg tissue normalized to amount of barcode injected) of lipid nanoparticles (LNP) of the invention versus control LNPs in: tumor (1A), brain (1B), kidneys (1C), spleen (1D), heart (1E) and lung (1F). The LNPs of the invention comprise an exemplary compound of the invention (LNP 16-25) and additional helper lipid(s). Control LNPs 2-15 have alternative ionizable lipids (e.g. D-Lin-MC3-DMA or DODAP), and Control LNP1 has a similar composition as the commercially available Onpattro, as disclosed in Example 1.

FIGS. 2A-2C are bar graphs representing in-vivo activity (of lipid nanoparticles (LNP)) comprising an exemplary compound of the invention as the ionizable lipid and encapsulating a reporter luciferase mRNA and a barcode, as disclosed in Example 2. FIG. 2A represents in-vivo activity of BN-INL-113 based LNP. FIG. 2B represents in-vivo activity of BN-INL-106 based LNP. FIG. 2C represents in-vivo activity of BN-INL-102 based LNP.

### DETAILED DESCRIPTION OF THE INVENTION

According to a first aspect, there is provided a compound comprising an ionizable moiety (e.g., head group) covalently bound to a lipophilic tail (e.g., hydrocarbon based chain), wherein the ionizable moiety is selected from (i) an ionizable moiety comprising a heteroaryl (e.g. imidazole); (ii) an ionizable moiety having a binding affinity to a CNS receptor; (iii) an ionizable moiety comprising a squaramide; and (iv) aza-crown ether. In some embodiments, the lipophilic tail comprises between 10 and 50 carbon atoms. In some embodiments, the compound is an amphiphilic compound. In some embodiments, the compound (optionally together with additional liposome forming lipid(s)) is capable of spontaneously self-assembling to form a nanoparticle (e.g., a liposome) in an aqueous solution.

As used herein, the term “liposome forming lipid” (also termed herein as “helper lipid”) encompasses lipids (e.g., phospholipids) which upon dispersion or dissolution thereof in an aqueous solution at a temperature above a transition temperature (Tₘ), undergo self-assembly so as to form stable vesicles (e.g., lipid nanoparticles). As used herein, the term Tm refers to a temperature at which the lipids undergo phase transition from solid (ordered phase, also termed as a gel phase) to a fluid (disordered phase, also termed as fluid crystalline phase). Tm also refers to a temperature (or to a temperature range) at which the maximal change in heat capacity occurs during the phase transition.

In some embodiments, the ionizable moiety is capable of undergoing ionization (protonation, or positive ionization) within a solution having a pH value below the pKa value of the ionizable moiety. In some embodiments, the ionizable moiety is capable of undergoing protonation within a solution having a pH value below the pKa value of the ionizable moiety. In some embodiments, at least 50 mol % of the ionizable moieties are positively charged (or protonated) within a solution having a pH value below the pKa value of the ionizable moiety.

In some embodiments, the pKa value of the ionizable moiety is between 5 and 7, including any range between. In some embodiments, the pKa value of the ionizable moiety is between 5 and 11, between 7 and 11, between 6 and 11, between 8 and 11, between 8 and 10, between 9 and 11, including any range between.

In some embodiments, the ionizable moiety comprises imidazole. In some embodiments, the ionizable moiety comprises histidine, including any salt and/or any derivative thereof. In some embodiments, the derivative of histidine comprises a decarboxylated histidine, an ester of histidine, a protected amine (amine protected by an amine protecting group), an imidazole bound to a spacer, or any combination thereof.

In some embodiments, the aza-crown ether comprises a macrocycle (e.g. between 9 and 18, or between 9 and 12 membered ring) comprising a plurality of nitrogen atoms (e.g. 3, 4, 5, 6 or more) and optionally one or more oxygen atoms.

In some embodiments, the ionizable moiety is bound to the lipophilic tail via a spacer or via a covalent bond.

In some embodiments, the lipophilic tail is an uncharged molecule. In some embodiments, the lipophilic tail is negatively charged. In some embodiments, the lipophilic tail (e.g. negatively charged lipophilic tail) comprises phosphate. In some embodiments, the lipophilic tail comprises a phosphodiester. In some embodiments, the lipophilic tail comprises one or more moieties represented by Formula I:

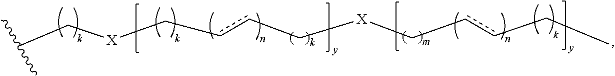

wherein:
wherein: [symbol] represents a single bond or a double bond; k, and y, are integers each independently being between 1 and 10; n and m are integers each independently being between 0 and 10; each X independently comprises (i) CH₂, CHR₁, NR₁, NH, O, S, or phosphate; or (ii)

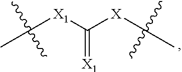

wherein each X₁ is independently O, NH, or S, and wherein X is CH₂, CHR₁, NR₁, NH, O, or S; and wherein R¹ comprises a C1-C20 alkyl, a C1-C20 alkenyl, a C20-C50 alkyl or alkenyl comprising one or more heteroatoms, or the hydrocarbon of Formula I. In some embodiments, the term “phosphate” refers to phosphodiester.

In some embodiments, the ionizable moiety has a MW of between 30 and 1,000 Da, between 30 and 100 Da, between 30 and 500 Da, between 30 and 300 Da, between 100 and 300 Da, between 100 and 500 Da, between 100 and 800 Da, between 300 and 500 Da, between 100 and 1,000 Da, between 500 and 800 Da, between 500 and 1,000 Da, between 800 and 1,000 Da, including any range between.

In some embodiments, the compound of the invention is represented by Formula 1:

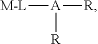

wherein: M represents an ionizable moiety; the ionizable moiety is selected from (i) an ionizable moiety comprising a heteroaryl; (ii) an ionizable moiety having a binding affinity to a CNS receptor; (iii) an ionizable moiety comprising a squaramide and (iv) a cyclic alkylamine ionizable moiety; L represents a spacer or is a bond; A represents a N, O, S, or C(H)₁₋₂ as allowed by valency; each R independently represents H, a hydrocarbon comprising between 10 and 50 carbon atoms, or is absent, and at least one R is the hydrocarbon; the hydrocarbon comprises a C20-C50 alkyl, a C20-C50 alkenyl, a C20-C50 alkyl or alkenyl comprising one or more heteroatoms.

In some embodiments, the ionizable moiety comprising a heteroaryl is any one of:

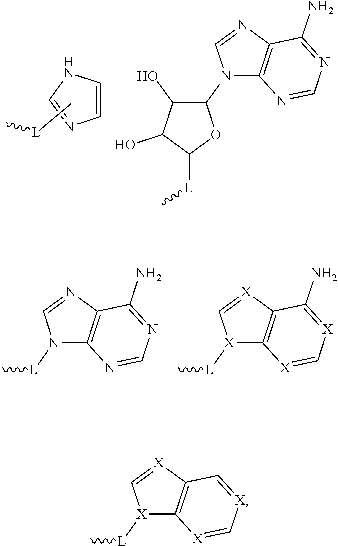

wherein L is as described herein, and each X is independently CH, CR1, or N, and wherein at least one X is N; and wherein the wavy bond is an attachment point to A.

In some embodiments, the ionizable moiety having a binding affinity to a CNS receptor is selected from glutathione, a methyl xanthine, purine (e.g. adenosine), or adenine, including any salt, any stereoisomer, and any tautomer thereof. In some embodiments, the ionizable moiety having a binding affinity to a CNS receptor is any of:

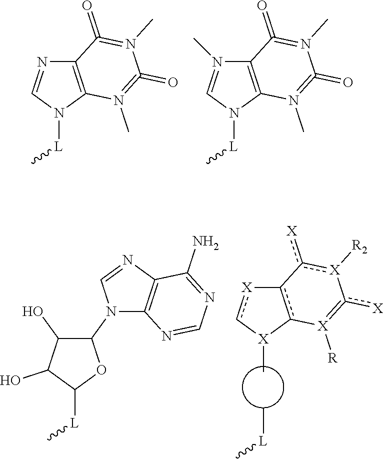

including any salt, any stereoisomer, and any tautomer thereof; wherein the wavy bond is an attachment point to A; wherein the circle represents an aliphatic or aromatic 5-6 membered ring, optionally substituted and optionally comprising a heteroatom (e.g. hexose, pentose, ribose, etc. including any derivative thereof such as tetrahydrofuran-3,4-diol); wherein R2 is H, is absent or a C1-C10 alkyl; wherein X and L are as described herein.

In some embodiments, the ionizable moiety comprising a squaramide (also termed as “squaramide-based ionizable moiety”) is

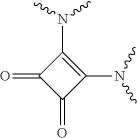

wherein each wavy bond is independently an attachment point to A, C1-C10 alkyl, or H, and wherein at least one wavy bond is the attachment point to A.

In some embodiments, the cyclic alkylamine ionizable moiety is any one of:

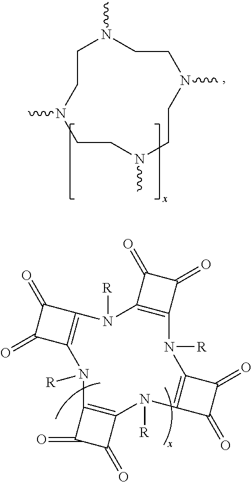

wherein x is between 1 and 4; and wherein each wavy bond is independently an attachment point to A or H, and wherein at least one wavy bond is the attachment point to A.

In some embodiments, the compound of the invention (e.g. the ionizable moiety) encompasses any of the compounds disclosed herein, including any salt, any solvate, any stereoisomer (e.g. enantiomer or diastereomer), and any tautomer (e.g. keto-enol, amide-iminol, or imine-enamine tautomer) thereof.

In some embodiments, the hydrocarbon is represented by Formula I:

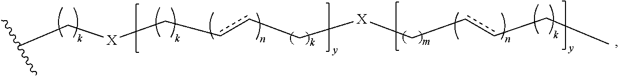

wherein: [symbol] represents a single bond or a double bond; k, and y, are integers each independently being between 1 and 10; n and m are integers each independently being between 0 and 10; each X independently comprises (i) CH2, CHR1, NR1, NH, O, S, or phosphate; or (ii)

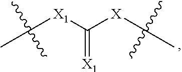

wherein each X1 is independently O, NH, or S, and wherein X is CH2, CHR1, NR1, NH, O, or S; and wherein R¹ comprises a C1-C20 alkyl, a C1-C20 alkenyl, a C20-C50 alkyl or alkenyl comprising one or more heteroatoms, or the hydrocarbon of Formula I. In some embodiments, R¹ is or comprises a hydrocarbon of Formula I:

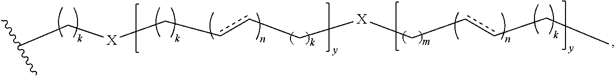

wherein: k, and y, are integers each independently being between 1 and 10; n and m are integers each independently being between 0 and 10; each X independently comprises (i) CH2, CHR1, NR1, NH, O, S, or phosphate; or (ii)

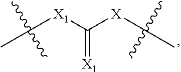

wherein each X1 is independently O, NH, or S, and wherein X is CH2, CHR1, NR1, NH, O, or S; and wherein R¹ comprises (i) a C1-C20 alkyl, (ii) a C1-C20 alkenyl, or (iii) a C20-C50 alkyl or alkenyl comprising one or more heteroatoms.

In some embodiments, the compound of the invention comprises (i) a squaramide-based ionizable moiety; or (ii) a cyclic alkylamine ionizable moiety; and wherein at least one hydrocarbon is as described herein, wherein at least one X is phosphate, and optionally wherein the additional X is an ester. In some embodiments, the compound of the invention comprises (i) a squaramide-based ionizable moiety; or (ii) a cyclic alkylamine ionizable moiety; and wherein at least one hydrocarbon is represented by Formula I, including any salt thereof; wherein X is ester; and wherein R1, k, y, n and m are as described hereinabove.

In some embodiments, the spacer comprises a first end covalently bound to A or to the lipid tail (represented by R), and a second end covalently bound to the ionizable moiety.

In some embodiments, the spacer is a small molecule, having a MW less than 1,000 Daltons (Da). In some embodiments, the spacer has a MW of between 100 and 1,000 Da, between 100 and 300 Da, between 100 and 500 Da, between 100 and 800 Da, between 300 and 500 Da, between 100 and 1,000 Da, between 500 and 800 Da, between 500 and 1,000 Da, between 800 and 1,000 Da, including any range between. Each possibility represents a separate embodiment.

In some embodiments, the spacer comprises an alkyl (e.g., C1-C10, or C₁-C₅ alkyl, including any range between), a glycol, an amide bond, an amine bond, an imine bond, an ether, an ester bond, a thioester bond, a disulfide bond, a natural and/or unnatural amino acid, a urea bond, including any derivative or a combination thereof.

In some embodiments, the compound of the invention is represented by Formula 1, wherein each R is or comprises any of:

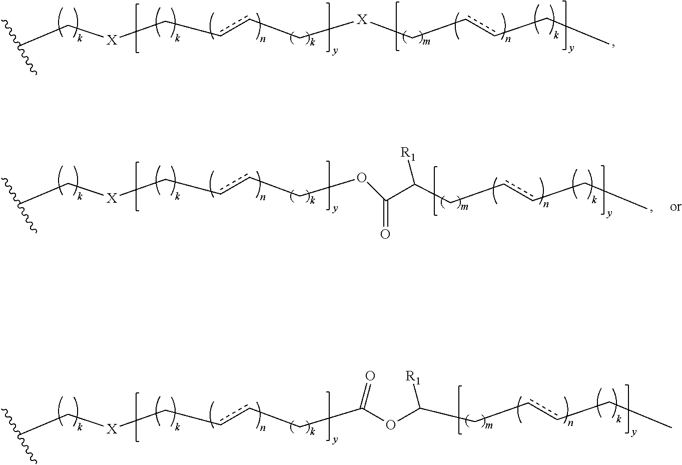

wherein X, R1, k, y, n and m are as described hereinabove.

In some embodiments, the compound of the invention is represented by Formula 1A:

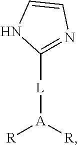

wherein each R is independently represented by Formula I, and wherein A and L are as described hereinabove.

In some embodiments, the compound of the invention is represented by Formula 1B:

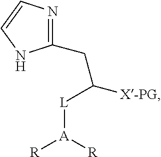

wherein X′ is or comprises O, N, NH, or S; wherein L, A, and R are as described hereinabove, and wherein PG represents a protecting group (N-protecting group). In some embodiments, L represents an alkyl, an ester, a carbonyl, an amide, or any combination thereof. In some embodiments, N-protecting groups or amino protecting groups are well-known in the art and include inter alia: acetyl, Fmoc, Alloc, Dde, iv-Dde, benzyl, benzyloxycarbonyl, tert-butyloxycarbonyl (Boc) and 2-[biphenylyl-(4)]-propyl-2-oxycarbonyl, dimethyl-3,5dimethoxybenzyloxycarbonyl, 2-(4-Nitrophenylsulfonyl) ethoxycarbonyl, 1,1-Dioxobenzo[b]thiophene-2-ylmethyloxycarbonyl, 2,7-Di-tert-butyl-Fmoc, 2-Fluoro-Fmoc, Nitrobenzenesulfonyl, Benzothiazole-2-sulfonyl, 2,2,2-Trichloroethyloxycarbonyl, Dithiasuccinoyl, and p-Nitrobenzyloxycarbonyl.

In some embodiments, the compound of the invention is represented by Formula 1B, wherein each R comprises

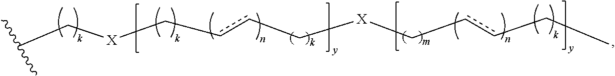

wherein X, k, y, n and m are as described hereinabove. In some embodiments, both R are the same or different.

In some embodiments, the compound of the invention is represented by Formula IC:

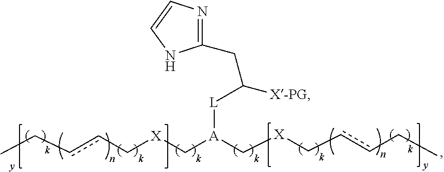

wherein X represents CH₂, CHR₁, NR₁, NH, O, S, or phosphate, and wherein R₁ comprises a C1-C20 alkyl, a C1-C20 alkenyl, a C20-C50 alkyl or alkenyl comprising one or more heteroatoms, and wherein k, y, n and m are as described hereinabove.

In some embodiments, the compound of the invention is represented by Formula 1B, wherein each R is or comprises any of:

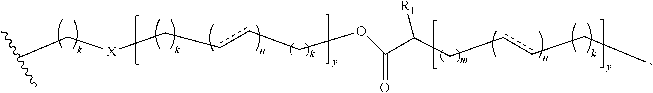

wherein X represents CH₂, CHR₁, NR₁, NH, O, S, or phosphate, and wherein R₁ comprises a C1-C20 alkyl, a C1-C20 alkenyl, a C20-C50 alkyl or alkenyl comprising one or more heteroatoms, and wherein k, y, n and m are as described hereinabove.

In some embodiments, the compound of the invention is or comprises any one of:

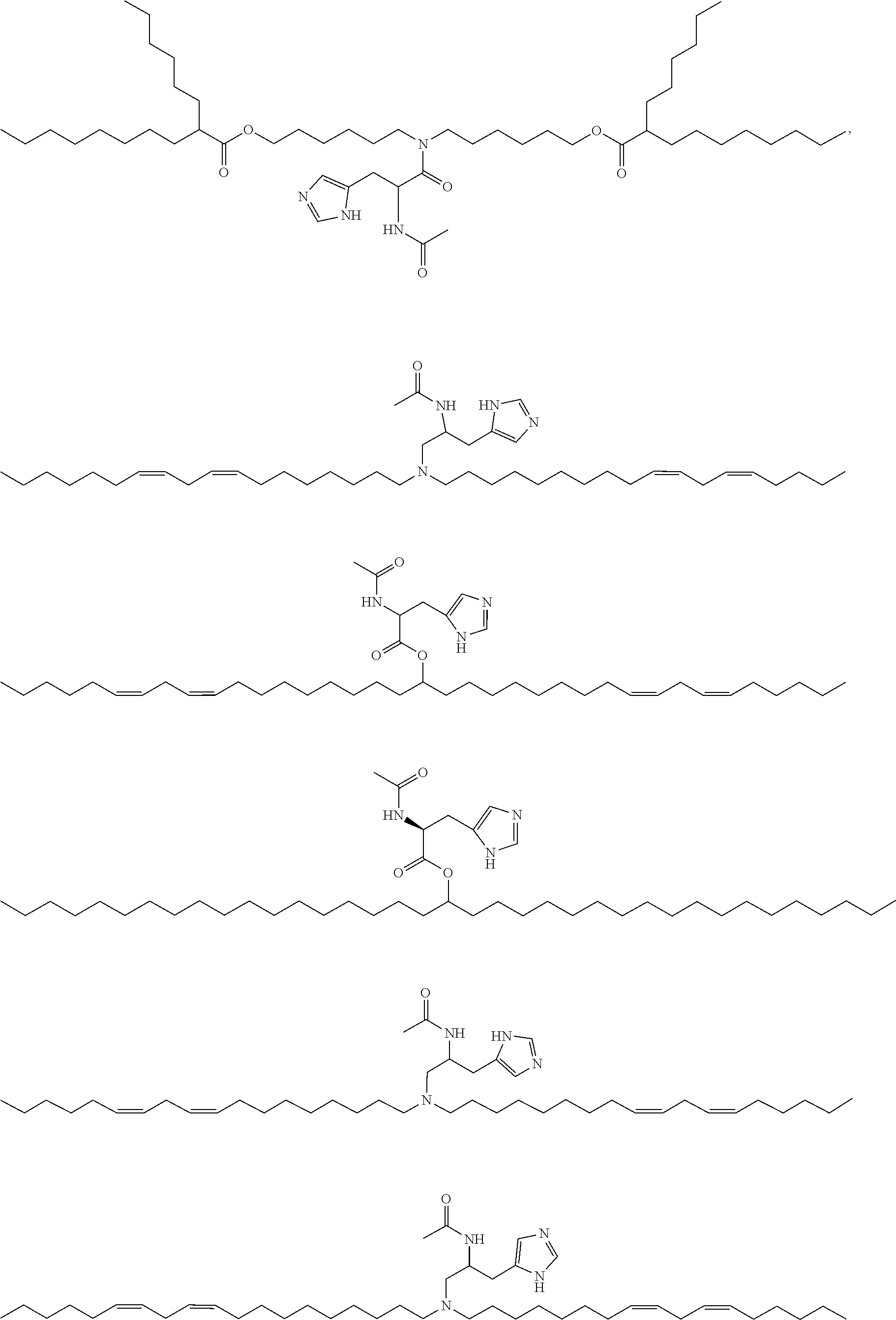

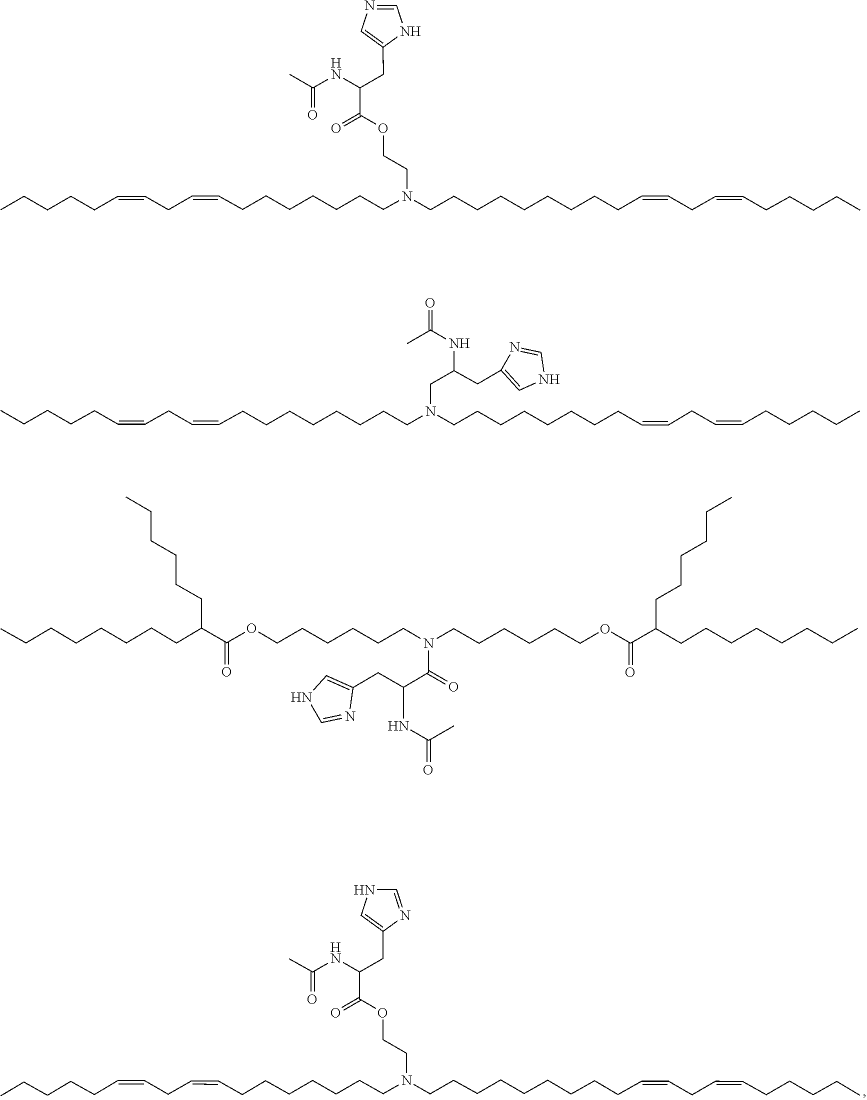

including any salt, any tautomer, and/or any stereoisomer (e.g., an enantiomer, and/or a diastereomer) thereof.

In some embodiments, the compound of the invention is represented by Formula 1, and each R is as described herein, and wherein the ionizable moiety is or comprises squaramide. In some embodiments, the ionizable moiety comprises a derivative of squaramide. In some embodiments, the squaramide derivative comprises an amine substituted by an alkyl, or a protecting group.

In some embodiments, the compound of the invention is or comprises

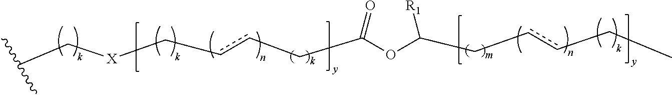

including any salt, and/or any tautomer thereof.

In some embodiments, the compound of the invention is represented by Formula 1, and each R is as described herein, and wherein the ionizable moiety has a binding affinity to a CNS receptor. In some embodiments, the ionizable moiety comprises a molecule capable of binding to one or more central nervous system (CNS) receptor. In some embodiments, binding is a reversible binding. In some embodiments, binding is a non-covalent binding. In some embodiments, the ionizable moiety is capable of binding to one or more CNS receptors, so as to cross the BBB and undergo internalization into a brain.

In some embodiments, a CNS receptor is located in a brain of a subject. In some embodiments, a CNS receptor is a transporter capable of internalizing the conjugate into the brain of a subject. In some embodiments, a CNS receptor is selected from: Adenosine receptor, GABA-transporter, Glucose transporter, N-methyl-d-aspartate (NMDA) receptor, α-amino-3-hydroxy-5-methyl-4-isoxazolepropionic acid (AMPA) receptor, Nicotinic cholinergic receptor, ASC transporter, 5-hydroxytryptamine receptor, serotonin receptor, Cannabinoid receptor, Dopamine receptor and norepinephrine transporter, including any tautomer, any stereoisomer (e.g. an enantiomer, or a diastereomer), an ester, an amide, or any combination thereof.

In some embodiments, the ionizable moiety comprises any of: adenine, adenosine, glutathione, Methylxanthine (e.g. caffeine), including any combination thereof.

In some embodiments, the compound of the invention is or comprises any one of:

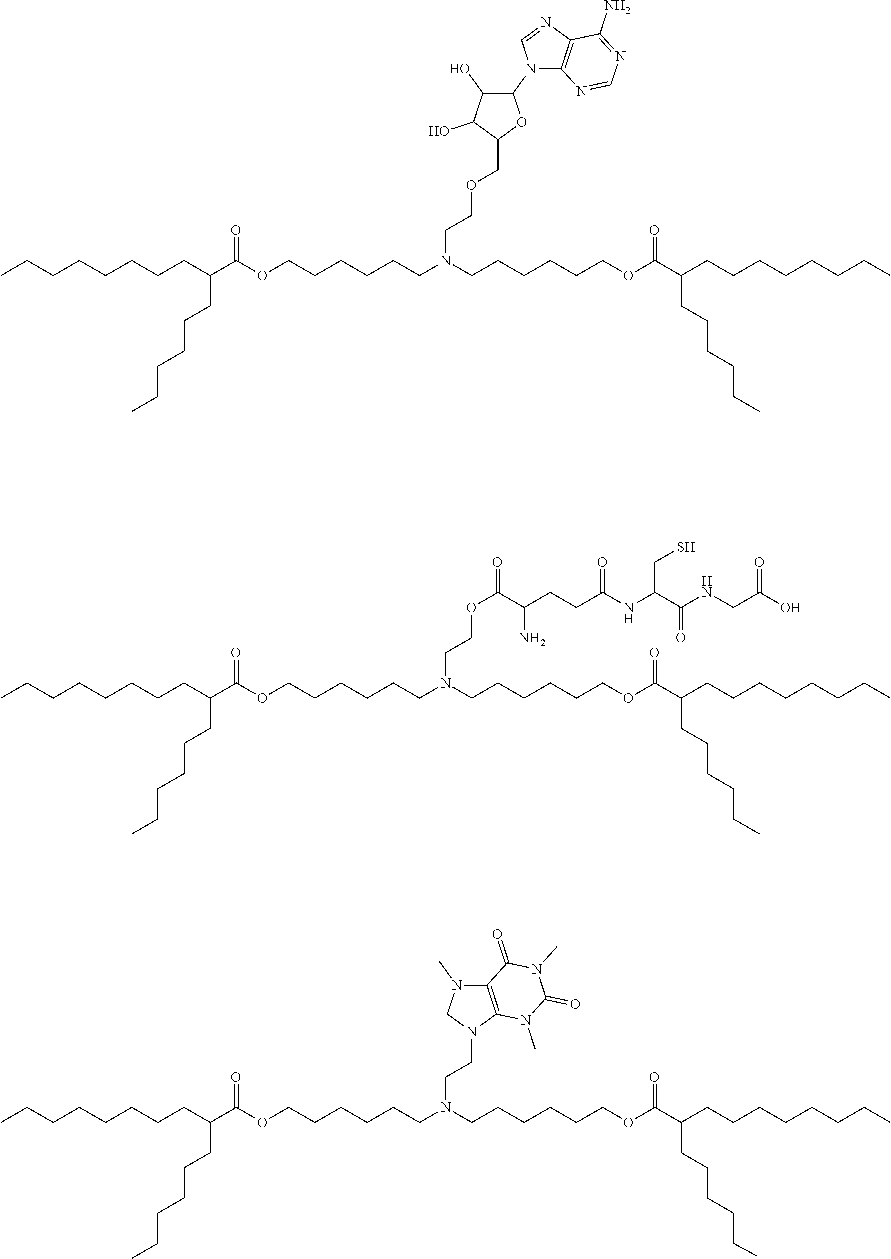

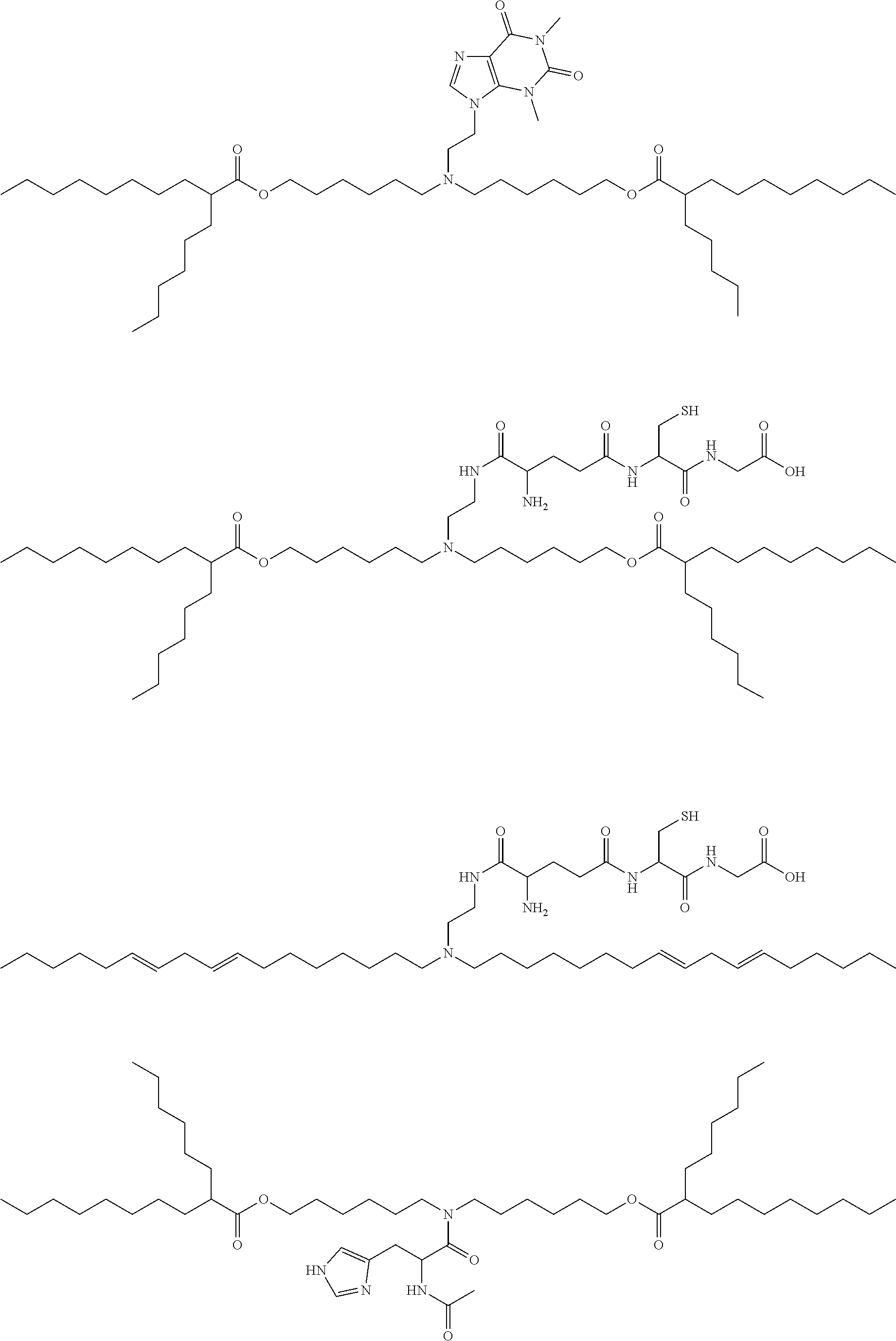

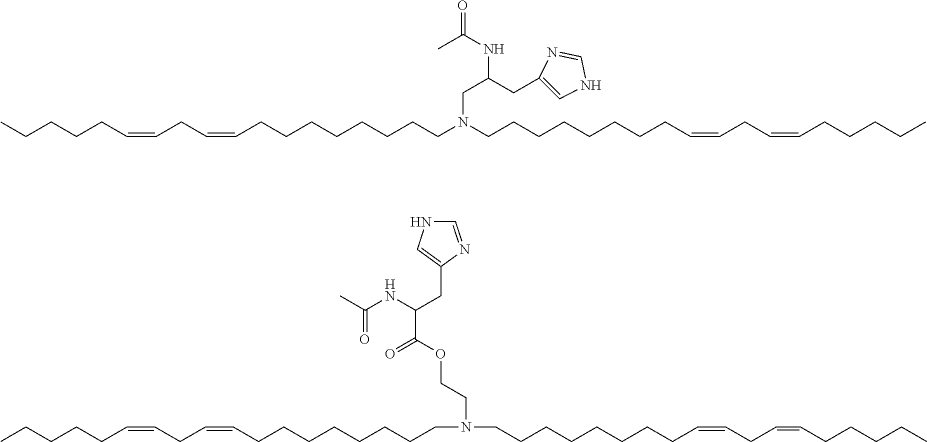

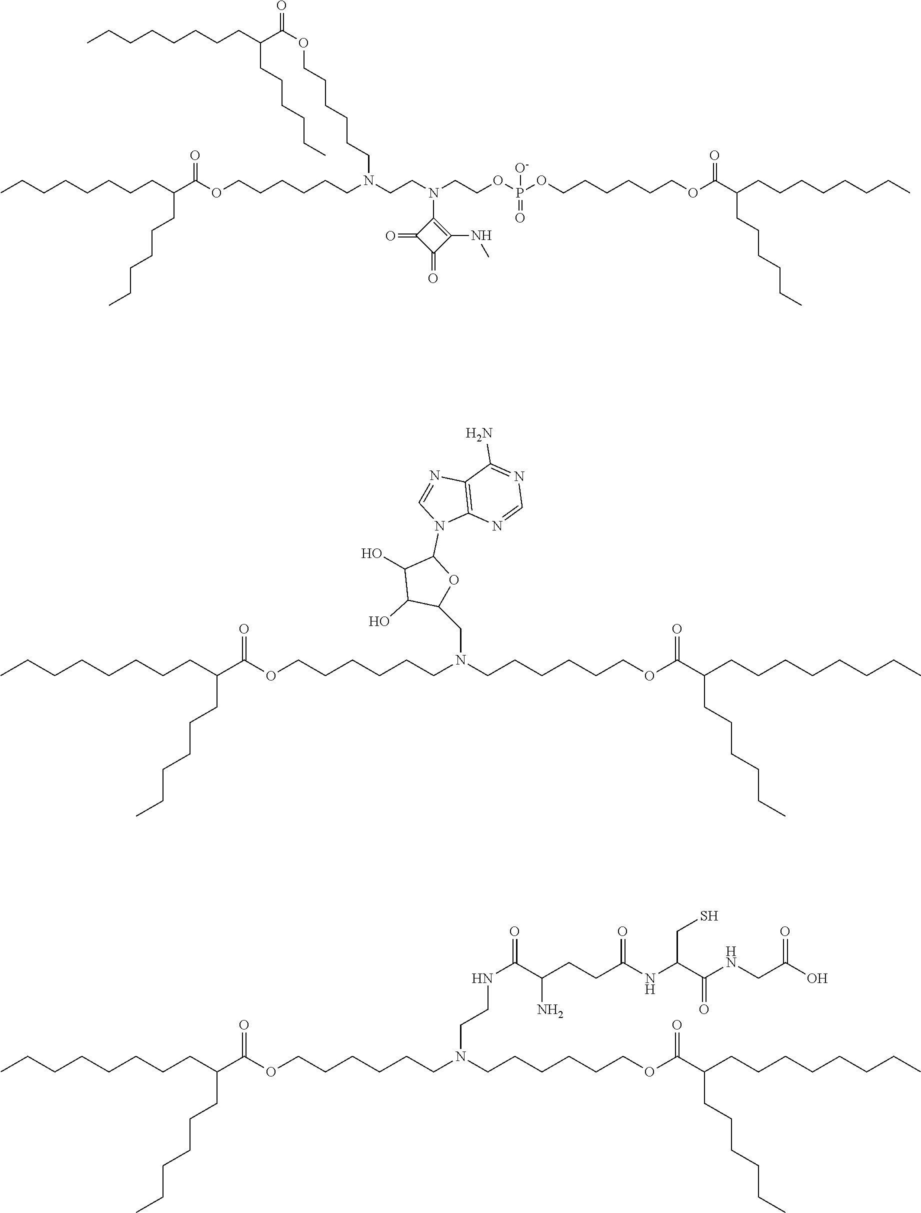

including any salt, any tautomer, any ester, any amide, and/or any stereoisomer thereof.

In some embodiments, the compound of the invention is represented by Formula 2:

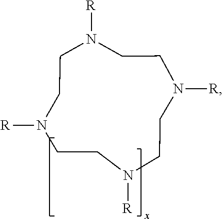

### or by Formula 3:

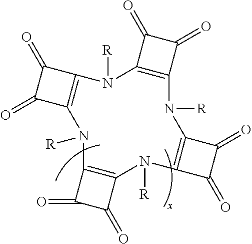

wherein x is between 1 and 4; and wherein each R is as described herein (e.g. comprises a C20-C50 alkyl, a C20-C50 alkenyl, a C20-C50 alkyl or alkenyl comprising one or more heteroatoms, is represented by Formula I, or is H) and at least one R is not H.

In some embodiments, the compound of the invention is or comprises any one of:

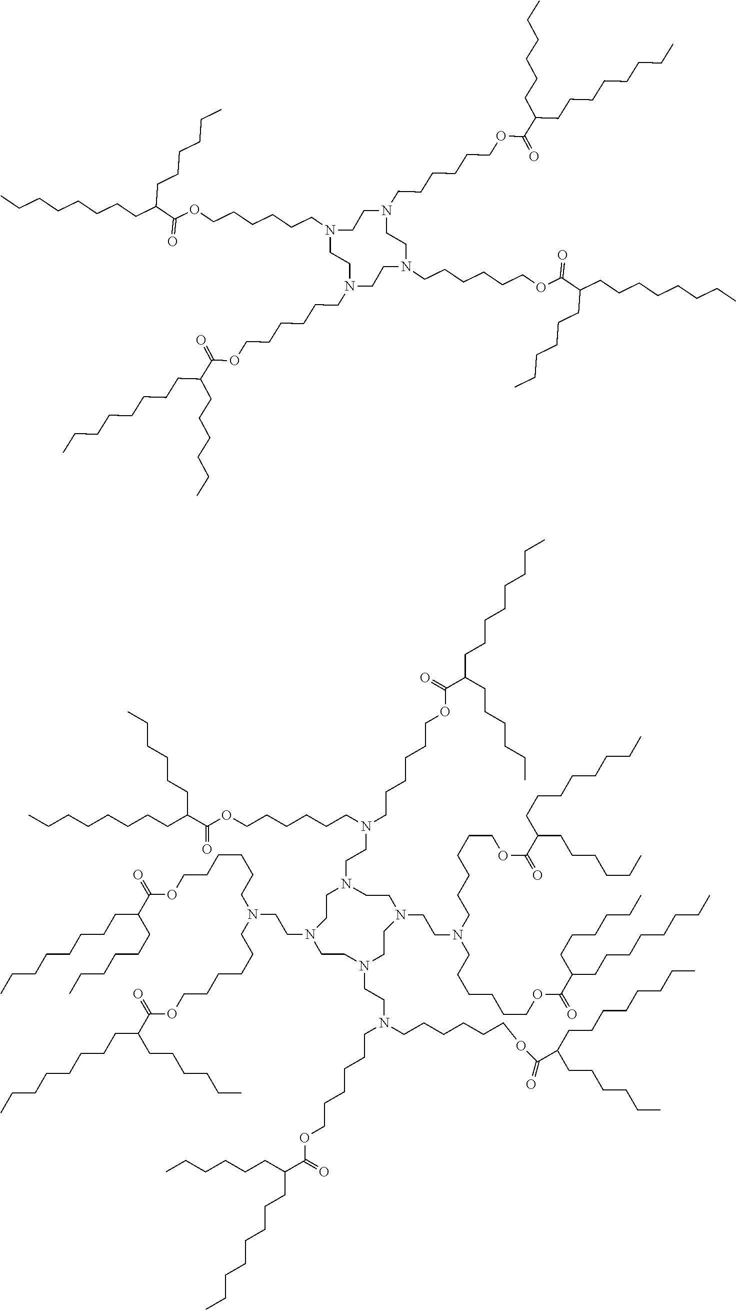

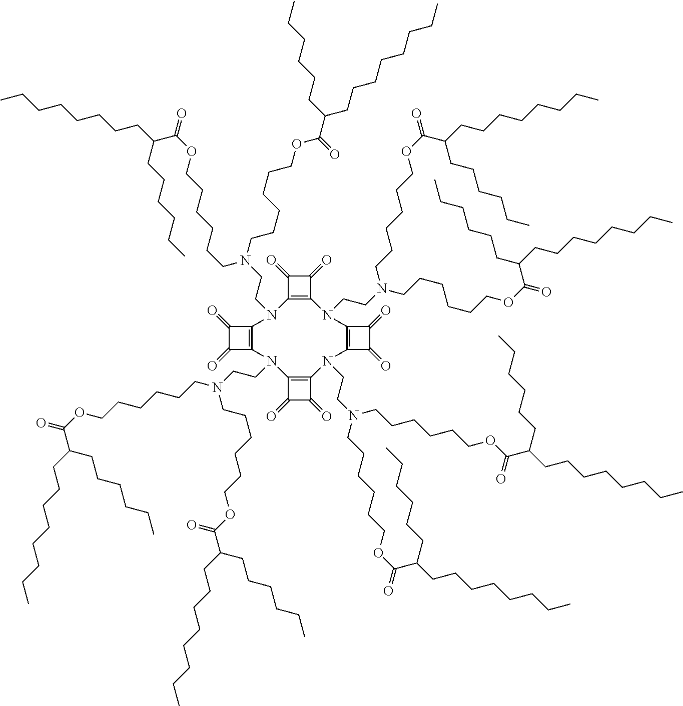 [SMILES: CCCCCCCCC(CCCCCC)C(=O)OCCCCCCN(CCCCCCOC(=O)C(CCCCCC)CCCCCCCC)CCN1c2c(c(=O)c2=O)N(CCN(CCCCCCOC(=O)C(CCCCCC)CCCCCCCC)CCCCCCOC(=O)C(CCCCCC)CCCCCCCC)c2c(c(=O)c2=O)N(CCN(CCCCCCOC(=O)C(CCCCCC)CCCCCCCC)CCCCCCOC(=O)C(CCCCCC)CCCCCCCC)c2c(c(=O)c2=O)N(CCN(CCCCCCOC(=O)C(CCCCCC)CCCCCCCC)CCCCCCOC(=O)C(CCCCCC)CCCCCCCC)c2c1c(=O)c2=O]

including any salt, any tautomer, any ester, any amide, and/or any stereoisomer thereof.

### Carriers

In another aspect, there is provided a carrier for an active agent(s), wherein the carrier is in a form of a core-shell nanoparticle comprising one or more compound(s) of the invention. In some embodiments, the carrier encapsulates the active agent within the core. In some embodiments, the active agent is a small molecule and/or a biologic molecule, such as polypeptide, a polynucleotide, etc. In some embodiments, the active agent is selected from a therapeutic agent, a prophylactic agent and a diagnostic agent including any combination thereof. In some embodiments, the one or more active agents are selected from the group consisting of: a protein, a peptide, a nucleic acid, a small molecule, a lipid, a glycolipid, and an antibody.

In some embodiments, the carrier is in the form of a lipid nanoparticle comprising the compound of the invention, and the active agent. In some embodiments, the lipid nanoparticle comprises a shell and an aqueous core, comprising the active agent. In some embodiments, the shell of the lipid nanoparticle comprises the compound of the invention. In some embodiments, the shell of the lipid nanoparticle further comprises a lipid, a sterol, and/or a modified lipid (e.g., PEG-lipid), or any combination thereof. In alternative embodiments, the carrier is in the form of a lipid nanoparticle comprising the compound of the invention, a lipid, and the active agent. In some embodiments, lipid nanoparticle comprises is in a form of a core-shell nanoparticle, wherein the shell of the nanoparticle comprises a lipid, and at least one compound of the invention. In some embodiments, the compound of the invention is bound (e.g., via electrostatic interactions) to the active agent (e.g., a polynucleotide).

In some embodiments, under suitable conditions the compound(s) of the invention, and optionally the lipid (and further optionally the active agent) spontaneously undergo self-assembly in an aqueous solution, so as to form the lipid nanoparticle. In some embodiments, the term “lipid nanoparticle” refers to a nanoparticle (e.g., substantially spherical particle), wherein the shell of the nanoparticle comprises one or more compounds of the invention and optionally one or more lipids (e.g., a helper lipid, such as a cationic lipid, non-cationic lipid; and optionally a sterol, and/or a PEG-modified lipid). Preferably, the lipid nanoparticles are formulated to deliver one or more active agents to one or more target cells.

In some embodiments, the nanoparticle has a spherical geometry or shape. In some embodiments, the nanoparticle has an inflated or a deflated shape. In some embodiments, a plurality of core-shell particles is devoid of any characteristic geometry or shape. In some embodiments, the nanoparticle has a spherical shape, a quasi-spherical shape, a quasi-elliptical sphere, a deflated shape, a concave shape, an irregular shape, or any combination thereof. In some embodiments, the nanoparticle has a spherical geometry or shape. In some embodiments, the nanoparticle has an inflated or a deflated shape. In some embodiments, a plurality of core-shell particles is devoid of any characteristic geometry or shape. In some embodiments, the nanoparticle has a spherical shape, a quasi-spherical shape, a quasi-elliptical sphere, a deflated shape, a concave shape, an irregular shape, or any combination thereof.

In alternative embodiments, the carrier is in the form of a core-shell nanoparticle comprising a shell (or a lipid membrane) encapsulating an aqueous core. In some embodiments, the shell is a multi-layered shell. In some embodiments, the shell of the nanoparticle comprises a lipid, and at least one compound of the invention. In some embodiments, under suitable conditions the lipid and at least one compound of the invention spontaneously undergo self-assembly in an aqueous solution, so as to form the core-shell nanoparticle. In some embodiments, the lipid is or comprises a phospholipid. In some embodiments, the lipid is or comprises a modified lipid (e.g., a modified phospholipid). In some embodiments, the lipid is or comprises a liposome forming lipid.

In some embodiments, the modified lipid is or comprises a PEG-lipid. In some embodiments, the PEG-lipid comprises a single PEG moiety covalently bound to the head group of the lipid. In some embodiments, the PEG moiety comprises an alkylated PEG such as methoxy poly(ethylene glycol) (mPEG). The PEG moiety can have a molecular weight of the head group from about 750 Da to about 20,000 Da, at times, from about 750 Da to about 12,000 Da and typically between about 1,000 Da to about 5,000 Da, including any range between.

In some embodiments, the shell further comprises a non-liposome forming lipid. When referring to a non-liposome forming lipid it is to be understood as referring to a lipid that does not spontaneously form into a vesicle when brought into an aqueous medium.

There are various types of lipids that do not spontaneously vesiculate and yet are used or can be incorporated into vesicles. In some embodiments, the non-liposome forming lipid is or comprises a sterol.

Non-limiting examples of sterols include but are not limited to: β-sitosterol, β-sitostanol, stigmasterol, stigmastanol, campesterol, campestanol, ergosterol, avenasterol, brassicasterol, fucosterol, cholesterol (CHOL), cholesteryl hemisuccinate, and cholesteryl sulfate including any salt or any combination thereof.

In some embodiments, the aqueous core comprises the active agent dissolved or dispersed therewithin. In some embodiments, the nanoparticle comprises a liposomal membrane (e.g., a lipid bilayer).

In some embodiments, a molar concentration of one or more compounds of the invention within the nanoparticle is between 3 and 80 mol %, between 3 and 60 mol %, between 5 and 60 mol %, between 10 and 20 mol %, between 20 and 60 mol %, between 10 and 60 mol %, between 20 and 40 mol %, between 40 and 60 mol %, between 60 and 80 mol %, including any range between. As used herein, the term “concentration” or “molar concentration” refers to a molar ratio relative to the total lipid content of the nanoparticle. In some embodiments, the total lipid content refers to the combined content of the compound of the invention and of the lipid, wherein the lipid encompasses a liposome forming lipid, a modified lipid, and a non-liposome forming lipid. In some embodiments, the total lipid content is substantially located within the shell of the carrier.

In some embodiments, the carrier is a lipid nanoparticle LNP comprising or consisting essentially of (i) a compound of the invention; (ii) a lipid, (iii) a sterol, and optionally (iv) a modified lipid. In some embodiments, a molar concentration of one or more compounds of the invention within the LNP is between 30 and 60 mol %, between 30 and 50 mol %, between 30 and 55 mol %, between 40 and 60 mol %, between 40 and 50 mol %, between 45 and 60 mol %, between 45 and 55 mol %, including any range between.

In some embodiments, a molar concentration of the lipid (i.e. helper lipid) within the nanoparticle is between 3 and 40 mol %, between 5 and 40 mol %, between 3 and 12 mol %, between 3 and 20 mol %, between 5 and 10 mol %, between 10 and 40 mol %, between 10 and 30 mol %, between 5 and 20 mol %, between 20 and 40 mol %, including any range between.

In some embodiments, a molar concentration of the helper lipid within the nanoparticle is between 3 and 40 mol %, between 5 and 40 mol %, between 3 and 12 mol %, between 3 and 20 mol %, between 5 and 30 mol %, between 5 and 10 mol %, between 5 and 15 mol %, between 5 and 20 mol %, between 5 and 25 mol %, between 10 and 30 mol %, between 10 and 20 mol %, between 10 and 25 mol %, including any range between.

In some embodiments, the helper lipid is or consist essentially of a phospholipid. In some embodiments, the helper lipid is or consist essentially of a compound of the invention (e.g. a compound comprising squaramide, as disclosed herein). In some embodiments, the helper lipid comprises a first helper lipid and a second helper lipid, wherein the first helper lipid and the second helper lipid is independently any of the lipids disclosed herein. In some embodiments, the combined molar concentration of the first helper lipid and of the second helper lipid within the LNP is between 5 and 20 mol %, between 5 and 30 mol %, between 5 and 25 mol %, between 10 and 20 mol %, between 10 and 25 mol %, between 10 and 15 mol %, including any range between. In some embodiments, the first helper lipid is a compound of the invention (e.g. a compound comprising squaramide, as disclosed herein). In some embodiments, the second helper lipid is a phospholipid (e.g. a zwitterionic phospholipid, such as DSPC). In some embodiments, a molar ratio between the first helper lipid and the second helper lipid within the LNP is between about 1:2 and about 1:3, including any range between.

In some embodiments, a molar concentration of the sterol within the nanoparticle is between 20 and 60 mol %, between 20 and 30 mol %, between 20 and 50 mol %, between 30 and 60 mol %, between 20 and 30 mol %, between 30 and 50 mol %, between 50 and 60 mol %, including any range between.

In some embodiments, a molar concentration of the sterol within the nanoparticle is between 25 and 50 mol %, between 25 and 30 mol %, between 25 and 35 mol %, between 25 and 45 mol %, between 25 and 40 mol %, between 30 and 50 mol %, between 30 and 45 mol %, between 25 and 40 mol %, including any range between.

In some embodiments, a molar concentration of the modified lipid (e.g., PEG-lipid) within the nanoparticle is between 0.5 and 10 mol %, between 0.1 and 10 mol %, between 0.1 and 0.5 mol %, between 0.5 and 1 mol %, between 1 and 5 mol %, between 5 and 10 mol %, between 5 and 7 mol %, between 7 and 10 mol %, 0.5 and 5 mol %, between 0.5 and 1 mol %, between 0.5 and 1.5 mol %, between 0.5 and 1.5 mol %, between 0.5 and 2 mol %, between 0.5 and 2.5 mol %, between 0.5 and 3 mol %, between 0.5 and 3.5 mol %, between 1 and 5 mol %, including any range between.

In some embodiments, the carrier is a lipid-based particle. In some embodiments, the carrier is a lipid nanoparticle (LNP). In some embodiments, the carrier is a liposome.

In some embodiments, the LNP is characterized by an average particle size below 300 nm, or between about 50 and about 250 nm and comprises or consists essentially of: (i) between about 30 and about 60 mol % of the compound of the invention; (ii) between about 25 and about 45 mol % of a sterol, (iii) between about 0.5 and about 5 mol % of a modified lipid, and optionally comprises (iv)) between about 5 and about 15 mol % of a helper lipid which is not the compound of the invention. In some embodiments, the compound of the invention is an ionizable lipid, a helper lipid (e.g. a compound comprising squaramide, as disclosed herein) or both within the LNP. In some embodiments, the LNP is devoid of a helper lipid which is not the compound of the invention.

In some embodiments, the LNP is an injectable particle, characterized by an average particle size below 300 nm, or between about 50 and about 250 nm. In some embodiments, the LNP comprises or consists essentially of: (i) between about 30 and about 60 mol % of the compound of the invention; (ii) between about 5 and about 30 mol %, or between about 10 and about 20 mol %, of a helper lipid, (iii) between about 25 and about 50 mol % of a sterol, and optionally (iv) between about 0.5 and about 5 mol % of a modified lipid. In some embodiments, the LNP further encapsulates the active agent (a polynucleic acid), and is characterized by N:P ratio between 3 and 12, between 4 and 6, between 4 and 10, between 4 and 8, including any range between.

In one embodiment, the carrier is characterized by an average particle size of less than 500 nm to facilitate its entrance through the extracellular matrix to a cell. In one embodiment, the carrier is characterized by an average particle size of less than 300 nm in diameter to facilitate its entrance through the extracellular matrix to a cell.

In one embodiment, the carrier is characterized by an average particle size of less than 300 nm, less than 250 nm, less than 200 nm, less than 150 nm, less than 100 nm, less than 50 nm, less than 20 nm, including any range between.

In another embodiment, the carrier is characterized by an average particle size of between 30 and 300 nm, between 30 and 50 nm, between 50 and 300 nm, between 50 and 250 nm, between 30 and 200 nm, between 50 and 200 nm, between 100 and 300 nm, between 50 and 100 nm, between 200 and 300 nm, including any range between.

In some embodiments, an average particle size of between 30 and 250 nm, between 30 and 50 nm, between 30 and 250 nm, between 30 and 150 nm, between 50 and 100 nm, between 50 and 150 nm, between 50 and 200 nm, between 50 and 250 nm, between 75 and 150 nm, between 75 and 225 nm, between 100 and 250 nm, including any range between.

In some embodiments, the carrier is characterized by a poly dispersity index (PDI) of less than 0.3, less than 0.28, less than 0.25, less than 0.2, and between 0.05 and 0.3, between 0.05 and 0.1, between 0.05 and 0.15, between 0.05 and 0.2, between 0.05 and 0.25, between 0.1 and 0.3, including any range in between.

As used herein, the average particle size and PDI values are determined by DLS.

In some embodiments, the active agent is encapsulated within the LNPs. In some embodiments, the concentration of the encapsulated active agent within a liquid composition comprising LNP is at least 1 ng/ul, at least 5 ng/ul, at least 10 ng/ul, at least 20 ng/ul, at least 21 ng/ul, at least 22 ng/ul, between 20 and 1000 ng/ul, including any range in between. In some embodiments, the encapsulated active agent concentration is between 10 and 200 ng/ul, between 20 and 200 ng/ul, between 20 and 100 ng/ul, between 20 and 50 ng/ul, between 50 and 250 ng/ul, between 25 and 75 ng/ul, between 50 and 150 ng/ul, between 100 and 1000 ng/ul, between 50 and 500 ng/ul, between 50 and 750 ng/ul, between 250 and 1000 ng/ul including any range in between.

In another embodiment, the carrier is characterized by a negative zeta potential (e.g., measure at a pH between about 6.5 and 7.5). In some embodiments, the carrier is characterized by a negative zeta potential ranging between −0.1 and −10 mV, including any range between. In some embodiments, the carrier is characterized by a positive zeta potential ranging between +0.1 and +10 mV, including any range between.

In some embodiments, the carrier is stable for a time period ranging between 1 day and 1 year, or more, including any range between. In some embodiments, the term “stable” refers to physical and chemical stability of the carrier (such as being substantially devoid of phase separation, agglomeration, disintegration, and/or substantially retaining the initial loading of the active agent) under appropriate storage conditions. In some embodiments, the term “stable” refers to physical and chemical stability of the carrier within an aqueous solution (e.g., dispersion stability).

In some embodiments, the morphology of the carrier may be spherical or substantially spherical, non-spherical (e.g., elliptical, tubular, etc.), irregular etc.

As used herein, the phrase “lipid nanoparticle” refers to a transfer vehicle, wherein the shell of the carrier comprises one or more lipids (e.g., liposome forming lipids also termed as “helper lipids”, such as cationic lipids, non-cationic lipids, and PEG-modified lipids) and/or one or more compounds of the invention. Furthermore, the lipid nanoparticles further comprise a non-liposome forming lipid, such as a sterol. Preferably, the lipid nanoparticles are formulated to deliver one or more agents to one or more target cells.

In some embodiments, the carrier comprises a non-cationic lipid, and the compound of the invention. In some embodiments, the carrier comprises a non-cationic lipid, the compound of the invention and a sterol. As used herein, the term “non-cationic lipid” refers to any neutral, or zwitterionic lipid. Non-cationic lipids include, but are not limited to, diolcoylphosphatidylethanolamine (DOPE), distearoylphosphatidylcholine (DSPC), hydrogenated soy phosphatidylcholine (HSPC), diolcoylphosphatidylcholine (DOPC), dipalmitoylphosphatidylcholine (DPPC), diolcoylphosphatidylglycerol (DOPG), dipalmitoylphosphatidylglycerol (DPPG), palmitoyloleoylphosphatidylcholine (POPC), palmitoyloleoyl-phosphatidylethanolamine (POPE), dioleoyl-phosphatidylethanolamine 4-(N-malcimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), or a mixture thereof.

In some embodiments, the carrier (e.g., a lipid nanoparticle) is prepared by combining an aqueous phase optionally comprising an active agent, and an organic phase comprising one or more lipid components and the compound of the invention. The selection of specific lipids (such as cationic lipids, non-cationic lipids, sterol(s) and/or PEG-modified lipids) which comprise the lipid nanoparticle, as well as the relative molar ratio of such lipids to each other and/or a molar ratio between the lipid(s) and the compound of the invention, is based upon the characteristics of the selected lipid(s), and the characteristics of the agents to be delivered. Additional considerations include, for example, the saturation of the alkyl chain, as well as the size, Tm, charge, pH, pKa, fusogenicity and toxicity of the selected lipid(s).

The inventors have surprisingly found that specific LNPs of the invention are characterized by distinct biodistribution profile in vivo, compared to similar LNPs devoid of the compound of the invention as the ionizable lipid or the helper lipid, or having a different ratio between the LNP constituents. As further demonstrated in the Examples section, some of the LNPs of the invention are characterized by an organ specific targeting (or affinity) and consequently by an enhanced accumulation within a specific organ as compared to other organs (such as liver) depending on the LNPs composition.

The term “specific targeting” or “affinity” as used herein encompasses the property of the LNP to undergo enhanced accumulation within a specific organ of a subject, as compared to a control, e.g., a similar LNP composition comprising an ionizable lipid which is not the compound of the invention (such as D-Lin-MC3-DMA).

In some embodiments, specific targeting comprises at least 2, at least 5, at least 10, at least 50, at least 100, at least 250, at least 500, at least 1000 times, or between 2 and 100, between 2 and 10, or between 2 and 1000 times greater accumulation of the LNP of the invention within the specific organ, as compared to a control. Accumulation of the LNP can be determined for example by analyzing expression of a specific RNA sequence (wherein the RNA is encapsulated within the LNP) within the specific organ, or by determining a signal (fluorescence, luminescence, etc.) emitted by a signal emitting probe encapsulated within LNP or translated from a specific encapsulated RNA sequence. Accumulation of the LNP can be determined for example by quantifying the number of copies of a barcode DNA encapsulated in the LNP.

In some embodiments, there is provided a LNP of the invention having specific affinity to a heart of a subject (also referred to as “heart specific LNP”) and is characterized by a molar concentration (i) of the compound of the invention of between 35 and 60%, between 35 and 55%, between 35 and 45%, between 45 and 55%, between 40 and 60%, including any range in between; (ii) of the helper lipid between 10 and 25%, between 10 and 20%, between 15 and 25%, between 15 and 20%, including any range in between; (iii) of the modified lipid of between 0.5 and 4.5%, between 0.5 and 1.5%, between 0.5 and 2.5%, between 1.5 and 3.5%, between 1.5 and 4.5%, including any range in between and (vi) of the sterol is between 25 and 45%, between 25 and 40%, between 30 and 40%, between 30 and 45%, including any range in between.

In some embodiments, the heart specific LNP composition is characterized by a molar concentration (i) of the compound of the invention of between 45 and 55%, between 47 and 55%, between 45 and 52%, between 49 and 51%, about 50%, including any range in between; (ii) of the helper lipid between 5 and 15%, between 7 and 15%, between 9 and 11%, between 8 and 12%, about 10%, including any range in between; (iii) of the modified lipid of between 1 and 2%, between 1.25 and 2%, between 1.25 and 1.75%, between 1 and 1.75%, about 1%, about 1.5%, about 2%, including any range in between and (vi) of the sterol is between 30 and 40%, between 35 and 40%, between 34 and 39%, between 30 and 39%, about 38%, about 39%, including any range in between.

In some embodiments, the heart specific LNP is as described hereinabove, wherein the compound of the invention is a compound of the invention, wherein the ionizable moiety comprises a heteroaryl. In some embodiments, the heart specific LNP is as described hereinabove, wherein the compound of the invention comprises a first compound and a second compound, wherein the first compound and the second compound comprises an ionizable moiety selected from heteroaryl-based ionizable moiety and squaramide-based ionizable moiety.

In some embodiments, the heart specific LNP comprises BN-INL-A106 and optionally further comprises BN-IPL-A111. In some embodiments, the heart specific LNP comprises about 50 mol % of BN-INL-A106, about 38-39 mol % of cholesterol, between about 1 and about 2 mol % of a PEG-lipid, and further comprises about 10 mol % of a helper lipid (e.g. a phospholipid, such as DOPE DOPC or DSPC and/or BN-IPL-A111). In some embodiments, the heart specific LNP comprises about 50 mol % of BN-INL-A106, about 38-39 mol % of cholesterol, between about 1 and about 2 mol % of a PEG-lipid, and further comprises about 10 mol % of a helper lipid (e.g. a phospholipid, such as DOPE DOPC or DSPC and/or BN-IPL-A111), and is characterized by a N:P ratio of 4.

In some embodiments, the heart specific LNP is or comprises BN-INL-A106 as the ionizable lipid. In some embodiments, the heart specific LNP is or comprises BN-IPL-A111 as the helper lipid. In some embodiments, the heart LNP specific is characterized by a N:P ratio of between about 4 and about 12, including any range between.

In some embodiments, there is provided an LNP of the invention having specific affinity to a tumor tissue (also referred to as “tumor specific LNP”), wherein a molar concentration of: (i) the compound of the invention is between 45 and 55%, between about 50 and 55%, between 48 and 52%, including any range in between; (ii) helper lipid is between 5 and 15%, between 7 and 12%, or about 10, including any range in between; (iii) modified lipid is between 1 and 2%, between 1.2 and 1.7%, about 1.5, including any range in between; and (iv) sterol is between 30 and 40%, between 35 and 40%, between about 36 and about 40, including any range in between. In some embodiments, the tumor specific LNP is as described hereinabove, wherein the compound of the invention is as disclosed herein, wherein the ionizable moiety has a binding affinity to a CNS receptor, as disclosed herein.

In some embodiments, the tumor specific LNP comprises or consists essentially of between 48 and 52 mol % of BN-INL-A113, between 7 and 12 mol % of the helper lipid, between 1 and 2 mol % of PEG-lipid, and between 35 and 40 mol % of cholesterol. In some embodiments, the tumor specific LNP is characterized by a N:P ratio of about 4.

In some embodiments, the tumor specific LNP comprises BN-INL-A113 as the ionizable lipid.

In some embodiments, there is provided an LNP of the invention having specific affinity to spleen (also referred to as “spleen specific LNP”), wherein a molar concentration of: (i) the compound of the invention is between 45 and 55%, between about 50 and 55%, between 48 and 52%, including any range in between; (ii) helper lipid is between 5 and 15%, between 7 and 12%, or about 10, including any range in between; (iii) modified lipid is between 1 and 2%, between 1.2 and 1.7%, about 1.5, including any range in between; and (iv) sterol is between 30 and 40%, between 35 and 40%, between about 36 and about 40, including any range in between, and wherein the compound of the invention is as disclosed herein, wherein the ionizable moiety comprises a cyclic alkylamine, as disclosed herein.

In some embodiments, the spleen specific LNP is as described hereinabove, wherein the compound of the invention is or comprises BN-INL-A120. In some embodiments, the spleen specific LNP is characterized by a N:P ratio of about 4.

In some embodiments, there is provided an LNP of the invention having specific affinity to liver (also referred to as “liver specific LNP”), wherein a molar concentration of: (i) the compound of the invention is between 45 and 55%, between about 50 and 55%, between 48 and 52%, including any range in between; (ii) helper lipid is between 3 and 13%, between 3 and 9%, or about 5, including any range in between; (iii) modified lipid is between 4 and 5%, about 4.5, including any range in between; and (iv) sterol is between 30 and 45%, between 35 and 42%, between about 35 and about 41, including any range in between, wherein the compound of the invention is a compound of the invention, wherein the ionizable moiety comprises a cyclic alkylamine as disclosed herein; and wherein the liver LNP composition is characterized by a N:P ratio of about 12.

In some embodiments, the liver LNP composition is or comprises BN-INL-A120 as the compound of the invention.

In some embodiments, there is provided an LNP of the invention having specific affinity to lungs (also referred to as “lung specific LNP”), wherein a molar concentration: (i) of the compound of the invention is between 30 and 40%, between 30 and 37%, between 33 and 40%, between 33 and 37%, about 35%, including any range in between; (ii) of the helper lipid between 20 and 30%, between 23 and 30%, between 20 and 27%, between 23 and 27%, including any range in between; (iii) of the modified lipid is between 1 and 2%, between 1.25 and 2%, between 1.25 and 1.75%, between 1 and 1.75%, including any range in between and (vi) of the sterol is between 37 and 41%, between 37 and 40%, between 38 and 40%, between 38 and 39%, including any range in between; and wherein the compound of the invention comprises the ionizable moiety comprising a heteroaryl, as disclosed herein; and wherein the lung specific LNP is characterized by a N:P ratio of about 9.

In some embodiments, the lung specific LNP is or comprises BN-INL-A102 as the compound of the invention.

In some embodiments, there is provided an LNP of the invention having specific affinity to the brain (also referred to as “brain specific LNP”), wherein a molar concentration: (i) of the compound of the invention is between 50 and 60%, between 53 and 60%, between 53 and 57%, between 50 and 60%, including any range in between; (ii) of the helper lipid is between 5 and 15%, between 8 and 15%, between 8 and 12%, between 5 and 12%, including any range in between; (iii) of the modified lipid is between 4 and 5%, between 4.25 and 5%, between 4.25 and 4.75%, between 4 and 4.75%, including any range in between and (iv) of sterol is between 25 and 35%, between 30 and 35%, between 28 and 32%, between 28 and 35%, including any range in between, wherein the brain specific LNP is characterized by a N:P ratio of about 3, and wherein the compound of the invention comprises the ionizable moiety having a binding affinity to a CNS receptor, as disclosed herein.

In some embodiments, brain LNP composition is or comprises BN-INL-A113 as the compound of the invention.

### Pharmaceutical Composition

In another aspect, there is provided a pharmaceutical composition comprising the lipid nanoparticles of the invention and a pharmaceutically acceptable carrier. In some embodiments, the pharmaceutically acceptable carrier is also referred to as an excipient or adjuvant. As used herein, the term “carrier,” “excipient,” or “adjuvant” refers to any component of a pharmaceutical composition that is not the active agent. As used herein, the term “pharmaceutically acceptable carrier” refers to non-toxic, inert solid, semi-solid liquid filler, diluent, encapsulating material, formulation auxiliary of any type, or simply a sterile aqueous medium, such as saline. Some examples of the materials that can serve as pharmaceutically acceptable carriers are sugars, such as lactose, glucose and sucrose, starches such as corn starch and potato starch, cellulose and its derivatives such as sodium carboxymethyl cellulose, ethyl cellulose and cellulose acetate; powdered tragacanth; malt, gelatin, talc; excipients such as cocoa butter and suppository waxes; oils such as peanut oil, cottonseed oil, safflower oil, sesame oil, olive oil, corn oil and soybean oil; glycols, such as propylene glycol, polyols such as glycerin, sorbitol, mannitol and polyethylene glycol; esters such as ethyl oleate and ethyl laurate, agar; buffering agents such as magnesium hydroxide and aluminum hydroxide; alginic acid; pyrogen-free water; isotonic saline, Ringer's solution; ethyl alcohol and phosphate buffer solutions, as well as other non-toxic compatible substances used in pharmaceutical formulations. Some non-limiting examples of substances which can serve as a carrier herein include sugar, starch, cellulose and its derivatives, powered tragacanth, malt, gelatin, talc, stearic acid, magnesium stearate, calcium sulfate, vegetable oils, polyols, alginic acid, pyrogen-free water, isotonic saline, phosphate buffer solutions, cocoa butter (suppository base), emulsifier as well as other non-toxic pharmaceutically compatible substances used in other pharmaceutical formulations. Wetting agents and lubricants such as sodium lauryl sulfate, as well as coloring agents, flavoring agents, excipients, stabilizers, antioxidants, and preservatives may also be present. Any non-toxic, inert, and effective carrier may be used to formulate the compositions contemplated herein. Suitable pharmaceutically acceptable carriers, excipients, and diluents in this regard are well known to those of skill in the art, such as those described in The Merck Index, Thirteenth Edition, Budavari et al., Eds., Merck & Co., Inc., Rahway, N.J. (2001); the CTFA (Cosmetic, Toiletry, and Fragrance Association) International Cosmetic Ingredient Dictionary and Handbook, Tenth Edition (2004); and the “Inactive Ingredient Guide,” U.S. Food and Drug Administration (FDA) Center for Drug Evaluation and Research (CDER) Office of Management, the contents of all of which are hereby incorporated by reference in their entirety. Examples of pharmaceutically acceptable excipients, carriers and diluents useful in the present compositions include distilled water, physiological saline, Ringer's solution, dextrose solution, Hank's solution, and DMSO. These additional inactive components, as well as effective formulations and administration procedures, are well known in the art and are described in standard textbooks, such as Goodman and Gillman's: The Pharmacological Bases of Therapeutics, 8th Ed., Gilman et al. Eds. Pergamon Press (1990); Remington's Pharmaceutical Sciences, 18th Ed., Mack Publishing Co., Easton, Pa. (1990); and Remington: The Science and Practice of Pharmacy, 21st Ed., Lippincott Williams & Wilkins, Philadelphia, Pa., (2005), each of which is incorporated by reference herein in its entirety. The presently described composition may also be contained in artificially created structures such as liposomes, ISCOMS, slow-releasing particles, and other vehicles which increase the half-life of the peptides or polypeptides in serum. Liposomes for use with the presently described peptides are formed from standard vesicle-forming lipids which generally include neutral and negatively charged phospholipids and a sterol, such as cholesterol. The selection of lipids is generally determined by considerations such as liposome size and stability in the blood. A variety of methods are available for preparing liposomes as reviewed, for example, by Coligan, J. E. et al, Current Protocols in Protein Science, 1999, John Wiley & Sons, Inc., New York, and see also U.S. Pat. Nos. 4,235,871, 4,501,728, 4,837,028, and 5,019,369.

The carrier may comprise, in total, from about 0.1% to about 99.99999% by weight of the pharmaceutical compositions presented herein.

In some embodiments, the pharmaceutical composition is formulated for systemic administration. In some embodiments, the pharmaceutical composition is formulated for administration to a subject. In some embodiments, the subject is a mammal. In some embodiments, the mammal is a human. In some embodiments, the mammal is a laboratory animal. Examples of laboratory animals include, but are not limited to, mice, rats, rabbits, hamsters, dogs, cats, and monkeys. In some embodiments, the mammal is a mouse or rat. In some embodiments, the subject is in need of the composition. In some embodiments, the subject is in need of treatment. In some embodiments, the subject is a volunteer for a diagnostic method. In some embodiments, the subject is in need of diagnosis.

In some embodiments, the pharmaceutical composition is a liquid composition. In some embodiments, the pharmaceutical composition is an injectable composition characterized by an average particle size of the LNPs in a range below 300 nm, or between about 50 and about 300, about 50 and about 250, about 100 and about 250 nm, including any range between. In some embodiments, the concentration of the encapsulated active agent within the pharmaceutical composition (i.e. injectable composition) is at least 5 ng/ul, at least 10 ng/ul, at least 20 ng/ul, at least 21 ng/ul, at least 22 ng/ul, between 20 and 1000 ng/ul, between 20 and 200 ng/ul, between 20 and 100 ng/ul, between 20 and 50 ng/ul, between 50 and 250 ng/ul, between 25 and 75 ng/ul, between 50 and 150 ng/ul, between 100 and 1000 ng/ul, between 50 and 500 ng/ul, between 50 and 750 ng/ul, between 250 and 1000 ng/ul, including any range in between. In some embodiments, the injectable composition is further characterized by viscosity at 20C of between 1 and 1000 cP, between 1 and 500 cP, between 1 and 300 cP, between 1 and 200 cP, between 1 and 100 cP, including any range in between.

In some embodiments, the pharmaceutical composition is for use in a therapeutic method. In some embodiments, a therapeutic method is a method of treatment. In some embodiments, the pharmaceutical composition is for use in a diagnostic method. In some embodiments, a diagnostic method is a method of diagnosing. In some embodiments, the pharmaceutical composition is for use in a theranostic method. In some embodiments, a theranostic method is a method of determining a suitable therapeutic for the subject. In some embodiments, the method comprises administering the composition of the invention to a subject.

As used herein, the terms “administering,” “administration,” and like terms refer to any method which, in sound medical practice, delivers a composition containing an active agent to a subject in such a manner as to provide a therapeutic effect. One aspect of the present subject matter provides for intravenous administration of a therapeutically effective amount of a composition of the present subject matter to a patient in need thereof. Other suitable routes of administration can include parenteral, subcutaneous, inhalation, oral, intramuscular, or intraperitoneal.

The dosage administered will be dependent upon the age, health, and weight of the recipient, kind of concurrent treatment, if any, frequency of treatment, and the nature of the effect desired.

In some embodiments, the composition administered is a composition described in international patent publication WO2016024281 and wherein the composition comprises a lipid of the invention or a nanoparticle of the invention. In some embodiments, the composition administered is a composition described in international patent publication WO2019008590 and wherein the composition comprises a lipid of the invention or a nanoparticle of the invention. In some embodiments, the compositions of the invention are for use in theranostic/diagnostic methods described in WO2016024281, herein incorporated by reference in its entirety. In some embodiments, the compositions of the invention are for use in the theranostic/diagnostic methods described in WO2019008590, herein incorporated by reference in its entirety. In some embodiments, the compositions of the invention are for use in predicting the response of a subject afflicted with a disease to at least one therapeutic agent. In some embodiments, the compositions of the invention are for use in predicting the response of a subject afflicted with a disease to a plurality of therapeutic agents.

### Active Agents

In some embodiments, the active agent is a diagnostic agent. In some embodiments, the active agent is a therapeutic agent.

In some embodiments, the diagnostic agent is a detectable agent, wherein detectable is by any suitable imaging techniques (e.g. MRI, X-ray, PET, SPECT, fluorescence imagining, etc.). In some embodiments, the diagnostic agent is elected from a diagnostic probe, a contrast agent, or both. In some embodiments, the diagnostic probe is selected from a fluorescent probe, an MRI probe (or MRI contrast agent), a radioactive isotope, or any combination thereof.

In some embodiments, the MRI probe is or comprises a magnetic metal. Any magnetic metal or a nanoparticle comprising thereof suitable for magnetic resonance imaging (MRI) may be used in the carrier and methods of the present invention. The MRI probe may yield a T2*, T2, or T1 magnetic resonance signals/signal contrast enhancement upon exposure to an external magnetic field. Examples of suitable magnetic metals include, but are not limited to magnetite, hematite, ferrites, and materials comprising one or more of iron, cobalt, manganese, nickel, chromium, gadolinium, neodymium, dysprosium, samarium, erbium, iron carbide, iron, and iron (III) oxide, including any salt, any chelate, any nanoparticle or any combination thereof.

In some embodiments, the diagnostic probe is a radiolabel. In some embodiments, the diagnostic probe is a radioactive isotope. In some embodiments, the diagnostic probe is a PET-tracer (i.e. a positron emitting isotope, such as C-11, F-18, Ga-68, Lu-177, Cu-64, etc., or a probe molecule labeled by a positron emitting isotope). In some embodiments, the PET-tracer is bound to the linker. In some embodiments, the PET-tracer is bound to the spacer. In some embodiments, the PET-tracer is a metal cation (e.g. Ga-68, Lu-177, Cu-64), wherein the PET-tracer is coordinatively bound to the chelator. In some embodiments, the PET-tracer comprises C-11 or F-18 covalently bound to a molecule (PET probe).

In some embodiments, the fluorescent probe comprises a fluorescent dye or a fluorophore. In some embodiments, the fluorescent probe is capable of emitting ultra-violet (UV) light. In some embodiments, the fluorescent probe is capable of emitting near infrared (IR) light. In some embodiments, the fluorescent probe is capable of emitting infrared light. In some embodiments, the fluorescent probe is capable of emitting visible light. In some embodiments, the fluorescent probe is capable of emitting UV, IR, near-IR and/or visible light. In some embodiments, the fluorescent probe is selected from, without being limited thereto, fluorescein, diacetylfluorescein, dipivaloyl Oregon green, cyanine dye (e.g. Cy-probes), porphyrin dye, red fluorescent probe Cy3, tetramethylrhodamine, far-red fluorescent Cy5, Alexa Fluor-dyes, Atto-dyes, BODIPY-dyes, Rhodamine-dyes (and Rhodamine silicone derivatives), GFP, enhanced GFP (cGFP), YFP, RFP, mCherry, Tomato and a quantum dot. In some embodiments, the fluorescent moiety is selected from GFP and Tomato.

In some embodiments, the contrast agent is a CT contrast agent. As will be apparent to those skilled in the art, any metal and/or combination of metals suitable for use for imaging by CT or X-ray may be used. In some embodiments, metals which can be used as a CT contrast agent are heavy metals, or metal with a high Z number. Examples of suitable metals include, but are not limited to: barium, gold, silver, platinum, palladium, cobalt, iron, copper, tin, tantalum, vanadium, molybdenum, tungsten, osmium, iridium, rhenium, hafnium, thallium, lead, bismuth, gadolinium, dysprosium, holmium, and uranium, or a combination thereof. In some embodiments, CT contrast agent is or comprises a halogen atom (e.g. Br, or I), or Ba-salt. I-based CT contrast agents are well known in the art such as Iohexol, or a Halo-tag.

In some embodiments, the sequence of the nucleic acid molecule (i.e., barcode) is exclusive of sequences, patterns, signatures or any other nucleic acid sequences associated with a material/substance/particle that is naturally occurring in the environment or particularly naturally occurring in the cell being targeted by the method and composition of the invention. In additional embodiments, the sequence of the nucleic acid molecule (i.e., barcode) is devoid of nucleotide sequences of more than 10 bases which can associate with a naturally occurring nucleotide sequence, and particularly of an exon. In another embodiment, the nucleic acid molecule comprises a sequence which is not substantially identical or complementary to the cell's genomic material (such as to prevent hybridization of the nucleic acid molecule with the cell's genomic material, particularly of the cell's exon and/or prevent false positive amplification results).

In some embodiments, a barcode is short. In some embodiments, short is less than or equal to 100, 90, 50, 45, 40, 35, 30, 25, 20, 15 or 10 bases. Each possibility represents a separate embodiment of the invention. In some embodiments, the barcode is not identical to or complementary to a sequence found in nature. In some embodiments, the barcode does not hybridize to a sequence found in nature. In some embodiments, found in nature is found in the subject. In some embodiments, found in nature is found in a target cell. Barcode sequences and molecules are well known in the art and are commercially available from numerous retailers.

A unique barcode (e.g., a nucleic acid having a unique sequence) is suitable for identifying at least one therapeutic agent within the carrier, or the composition of the carrier itself, after implementing the methods of the invention. Methods for the detection of the presence and identification of a nucleic acid sequence are known to a skilled artisan and include sequencing, ddPCR, and array (e.g., microarray) systems capable of enhancing the presence of multiple barcodes (e.g., commercially available by Ilumina Inc.).

In some embodiments, the composition comprises a plurality of types of nanoparticles. In some embodiments, a plurality is at least 2. In some embodiments, a plurality is at least 2, 3, 4, 5, 6, 7, 9, 10, 20, 25, 30, 40, 50, 75, 100, 125, 150, 175, 200, 225, 250, 275, 300, 350, 400, 450 or 500. Each possibility represents a separate embodiment of the invention. In some embodiments, each type of nanoparticle is different from the other. In some embodiments, the plurality comprises at least two nanoparticle types that differ from each other. In some embodiments, different is different in composition. In some embodiments, composition is lipid composition. In some embodiments, different is different in active agent. In some embodiments, different is different in dose of active agent. In some embodiments, different is different in nucleic acid molecule. In some embodiments, different in nucleic acid molecule is different in sequence. In some embodiments, the nucleic acid molecule uniquely identifies the type of nanoparticle.

In some embodiments, the therapeutic agent is water-soluble. In some embodiments, the therapeutic agent is characterized by water-solubility (e.g., at a temperature of between 10 and 50° C.) of at least 0.5 g/l, at least 1 g/l, at least 5 g/l, at least 10 g/l, at least 20 g/l, at least 50 g/l, and up to 200 g/l, up to 100 g/l, including any range between. In some embodiments, the therapeutic agent is or comprises a small molecule (e.g., an organic molecule having a molecular weight below 1000 Da, or below 500 Da, or between 100 and 1000, between 100 and 500 Da, including any range between). In some embodiments, the therapeutic agent is or comprises a biopolymer (e.g., polynucleic acid, polyamino acid, polysaccharide, or any combination or a copolymer thereof).

As used herein, the terms “peptide”, “polypeptide” and “polyamino acid” are used interchangeably and refer to a polymer of amino acid residues.

The terms “peptide”, “polypeptide” and “protein” as used herein encompass native peptides, peptide derivatives such as beta peptides, peptidomimetics (typically including non-peptide bonds or other synthetic modifications,) and the peptide analogs peptoids and semi-peptoids or any combination thereof. In another embodiment, the terms “peptide”, “polypeptide” and “polyamino acid” apply to amino acid polymers in which at least one amino acid residue is an artificial chemical analog of a corresponding naturally occurring amino acid.

The term “derivative” or “chemical derivative” includes any chemical derivative of the polypeptide having one or more residues chemically derivatized by reaction on the side chain or on any functional group within the peptide. Such derivatized molecules include, for example, peptides bearing one or more protecting groups (e.g., side chain protecting group(s) and/or N-terminus protecting groups), and/or peptides in which free amino groups have been derivatized to form amine hydrochlorides, p-toluene sulfonyl groups, carbobenzoxy groups, t-butyloxycarbonyl groups, acetyl groups or formyl groups. Free carboxyl groups may be derivatized to form amides thereof, salts, methyl and ethyl esters or other types of esters or hydrazides. Free hydroxyl groups may be derivatized to form O-acyl or O-alkyl derivatives. The imidazole nitrogen of histidine may be derivatized to form N-im-benzylhistidine. Also included as chemical derivatives are those peptides, which contain one or more naturally occurring amino acid derivatives of the twenty standard amino acid residues. For example: 4-hydroxyproline may be substituted for proline; 5-hydroxylysine may be substituted for lysine; 3-methylhistidine may be substituted for histidine; homoserine may be substituted for serine; and Dab, Daa, and/or ornithine (O) may be substituted for lysine.

In addition, a peptide derivative can differ from the natural sequence of the peptide of the invention by chemical modifications including, but are not limited to, terminal-NH2 acylation, acetylation, or thioglycolic acid amidation, and by amidation of the terminal and/or side-chain carboxy group, e.g., with ammonia, methylamine, and the like. Peptides can be either linear, cyclic, or branched and the like, having any conformation, which can be achieved using methods known in the art.

The term “amino acid” as used herein means an organic compound containing both a basic amino group and an acidic carboxyl group. Included within this term are naturally occurring amino acids, protected amino acids (e.g. comprising one or more protecting groups at the carboxyl, at the amine, and/or at the side chain of the amino acid), unusual, non-naturally occurring amino acids, as well as amino acids which are known to occur biologically in free or combined form but usually do not occur in proteins. Included within this term are modified and unusual amino acids, such as those disclosed in, for example, Roberts and Vellaccio (1983) The Peptides. 5:342-429. Modified, unusual or non-naturally occurring amino acids include, but are not limited to, D-amino acids, hydroxylysine, 4-hydroxyproline, N-Cbz-protected aminovaleric acid (Nva), ornithine (O), aminooctanoic acid (Aoc), 2,4-diaminobutyric acid (Abu), homoarginine, norleucine (Nle), N-methylaminobutyric acid (MeB), 2-naphthylalanine (2Np), aminoheptanoic acid (Ahp), phenylglycine, β-phenylproline, tert-leucine, 4-aminocyclohexylalanine (Cha), N-methyl-norleucine, 3,4-dehydroproline, N,N-dimethylaminoglycine, N-methylaminoglycine, 4-aminopipetdine-4-carboxylic acid, 6-aminocaproic acid, trans-4-(aminomethyl)-cyclohexanecarboxylic acid, 2-,3-, and 4-(aminomethyl)-benzoic acid, 1-aminocyclopentanecarboxylic acid, 1-aminocyclopropanecarboxylic acid, cyano-propionic acid, 2-benzyl-5-aminopentanoic acid, Norvaline (Nva), 4-O-methyl-threonine (TMe), 5-O-methyl-homoserine (hSM), tert-butyl-alanine (tBu), cyclopentyl-alanine (Cpa), 2-amino-isobutyric acid (Aib), N-methyl-glycine (MeG), N-methyl-alanine (MeA), N-methyl-phenylalanine (MeF), 2-thienyl-alanine (2Th), 3-thienyl-alanine (3Th), O-methyl-tyrosine (YMe), 3-Benzothienyl-alanine (Bzt) and D-alanine (DAI).

In some embodiments, the active agent comprises a therapeutic sequence. As used herein, the term “therapeutic sequence” refers to a polyamino acid or a polynucleic acid configured for inducing a therapeutic effect within a subject (e.g., treating, preventing, reducing symptoms of a disease, etc.). Further, the term “therapeutic sequence” encompasses any polyamino acid sequence or a polynucleic acid sequence capable of modifying the activity, functionality, survival, fitness, appearance, structure, development, behavior of a cell, or any combination thereof. In some embodiments, the therapeutic sequence is capable of binding an intracellular target, so as to control (upregulate, or downregulate) the activity of the intracellular target. In some embodiments, the intracellular target is selected from an intracellular protein, enzyme, receptor, RNA molecule, DNA molecule, or any combination thereof. In some embodiments, the therapeutic sequence is capable of binding to a target RNA/DNA sequence within a cell, so as to control expression of a gene of interest.

In some embodiments, the term “polynucleic acid” and the term “polynucleotide” are used herein interchangeably. In some embodiments, the polynucleotide comprises 60 to 15000 nucleobases, 10000 to 15000, 4700 to, 10000 200 to 5000 nucleobases, 300 to 5000 nucleobases, 400 to 5000 nucleobases, 400 to 2500 nucleobases, 200 to 3000 nucleobases, 400 to 2000 nucleobases, 400 to 1000 nucleobases, including any range between.

In some embodiments, the polynucleotide comprises at least 20 nucleobases, at least 250 nucleobases, at least 300 nucleobases, at least 350 nucleobases, at least 400 nucleobases, at least 450 nucleobases, at least 475 nucleobases, or at least 500 nucleobases. Each possibility represents a separate embodiment of the invention.

In some embodiments, the polynucleotide comprises 500 nucleobases at most, 750 nucleobases at most, 1,000 nucleobases at most, 1,250 nucleobases at most, 1,750 nucleobases at most, 2,500 nucleobases at most, 3000 nucleobases at most, 4000 nucleobases at most, or 5000 nucleobases at most. Each possibility represents a separate embodiment of the invention.

In some embodiments, the polynucleotide comprises a plurality of polynucleotide types. In some embodiments, the nanoparticle comprises a plurality of polynucleotide types. In some embodiments, the composition comprises a plurality of nanoparticle types, each type of nanoparticle comprises a specific polynucleotide.

In some embodiments, a specific polynucleotide comprises a plurality of polynucleotide molecules harboring the same or an identical nucleic acid sequence. In some embodiments, a specific polynucleotide comprises a plurality of polynucleotide molecules harboring essentially the same nucleic acid sequence.

As used herein, the term “plurality” encompasses any integer equal to or greater than 2. In some embodiments, a plurality comprises at least 2, at least 3, at least 4, at least 5, at least 6, at least 7, at least 8, at least 9, or at least 10, or any value and range therebetween. Each possibility represents a separate embodiment of the invention.

As used herein, the term “polynucleotide types” refers to a plurality of polynucleotides each of which comprises a nucleic acid sequence differing from any one of the other polynucleotides of the plurality of polynucleotides by at least 1 nucleobase, by at least 2 nucleobases, by at least 5 nucleobases, or by at least 10 nucleobases, or any value and range therebetween. Each possibility represents a separate embodiment of the invention.

In some embodiments, a polynucleotide comprises RNA, DNA, a synthetic analog of RNA, a synthetic analog of DNA, DNA/RNA hybrid, or any combination thereof. In some embodiments, a nanoparticle of the invention comprises a polynucleotide selected from: RNA, DNA, a synthetic analog of RNA, a synthetic analog of DNA, DNA/RNA hybrid, or any combination thereof.

In some embodiments, the polynucleotide comprises or consists of RNA. The polynucleotide comprises or consists of a messenger RNA (mRNA). “Messenger RNA” (mRNA) refers to any polynucleotide that encodes a (at least one) polypeptide (a naturally occurring, non-naturally occurring, or modified polymer of amino acids) and can be translated to produce the encoded polypeptide in vitro, in vivo, in situ or ex vivo. The basic components of an mRNA molecule typically include at least one coding region, a 5′ untranslated region (UTR), a 3′ UTR, a 5′ cap and a poly-A tail. Polynucleotides may function as mRNA but can be distinguished from wild-type mRNA in their functional and/or structural design features which serve to overcome existing problems of effective polypeptide expression using nucleic-acid based therapeutics.

The mRNA, as provided herein, comprises at least one (one or more) ribonucleic acid (RNA) polynucleotide having an open reading frame encoding at least one polypeptide of interest. In some embodiments, an RNA polynucleotide of an mRNA encodes 1-2, 2-10, 2-9, 2-8, 2-7, 2-6, 2-5, 2-4, 2-3, 3-10, 3-9, 3-8, 3-7, 3-6, 3-5, 3-4, 4-10, 4-9, 4-8, 4-7, 4-6, 4-5, 5-10, 5-9, 5-8, 5-7, 5- 6, 6-10, 6-9, 6-8, 6-7, 7-10, 7-9, 7-8, 8-10, 8-9 or 9-10 polypeptides. In some embodiments, an RNA polynucleotide of an mRNA encodes at least 10, 20, 30, 40, 50, 60, 70, 80, 90 or 100 polypeptides. In some embodiments, an RNA polynucleotide of an mRNA encodes at least 100 or at least 200 polypeptides.

In some embodiments, the nucleic acids are therapeutic mRNAs. As used herein, the term “therapeutic mRNA” refers to an mRNA that encodes a therapeutic protein. Therapeutic proteins mediate a variety of effects in a host cell or a subject in order to treat a disease or ameliorate the signs and symptoms of a disease. For example, a therapeutic protein can replace a protein that is deficient or abnormal, augment the function of an endogenous protein, provide a novel function to a cell (e.g., inhibit or activate an endogenous cellular activity, or act as a delivery agent for another therapeutic compound (e.g., an antibody-drug conjugate). Therapeutic mRNA may be useful for the treatment of the following diseases and conditions: bacterial infections, viral infections, parasitic infections, cell proliferation disorders, genetic disorders, and autoimmune disorders.

Thus, the structures of the invention can be used as therapeutic or prophylactic agents. They are provided for use in medicine. For example, the mRNA of the structures described herein can be administered to a subject, wherein the polynucleotides are translated in vivo to produce a therapeutic peptide.

In some embodiments, the polynucleotide comprises an inhibitory nucleic acid. In some embodiments, the polynucleotide comprises an oligonucleotide, such as antisense oligonucleotide.

As used herein, an “antisense oligonucleotide” refers to a nucleic acid sequence that is reversed and complementary to a DNA or RNA sequence.

As referred to herein, a “reversed and complementary nucleic acid sequence” is a nucleic acid sequence capable of hybridizing with another nucleic acid sequence comprised of complementary nucleotide bases. By “hybridize” is meant pair to form a double-stranded molecule between complementary nucleotide bases (e.g., adenine (A) forms a base pair with thymine (T) (or uracil (U) in the case of RNA), and guanine (G) forms a base pair with cytosine (C)) under suitable conditions of stringency. (See, e.g., Wahl, G. M., and S. L. Berger (1987) Methods Enzymol. 152:399; Kimmel, A. R. (1987) Methods Enzymol. 152:507). For the purposes of the present methods, the inhibitory nucleic acid need not be complementary to the entire sequence, only enough of it to provide specific inhibition; for example, in some embodiments the sequence is 100% complementary to at least nucleotides (nts) 2-7 or 2-8 at the 5′ end of the microRNA itself (e.g., the ‘seed sequence’), e.g., nts 2-7 or 20.

In some embodiments, the inhibitory nucleic acid has one or more chemical modifications to the backbone or side chains. In some embodiments, the inhibitory nucleic acid has at least one locked nucleotide, and/or has a phosphorothioate backbone.

Non-limiting examples of inhibitory nucleic acids useful according to the herein disclosed invention include, but are not limited to: antisense oligonucleotides, ribozymes, external guide sequence (EGS) oligonucleotides, siRNA compounds, single- or double-stranded RNA interference (RNAi) compounds such as siRNA compounds, modified bases/locked nucleic acids (LNAs), antagomirs, peptide nucleic acids (PNAs), ribozymes (catalytic RNA molecules capable to cut other specific sequences of RNA molecules) and other oligomeric compounds or oligonucleotide mimetics which hybridize to at least a portion of the target nucleic acid and modulate its function. In some embodiments, the inhibitory nucleic acids include antisense RNA, antisense DNA, chimeric antisense oligonucleotides, antisense oligonucleotides comprising modified linkages, interference RNA (RNAi), short interfering RNA (siRNA); a micro-RNA (miRNA); a small, temporal RNA (stRNA); or a short, hairpin RNA (shRNA); small RNA-induced gene activation (RNAa); small activating RNAs (saRNAs), or combinations thereof.

As used herein, the term “antisense oligonucleotide” refers to a polynucleotide molecule comprising 3-180 bases. In another embodiment, the term “antisense oligonucleotide” refers to a polynucleotide molecule of between 5-100, 5-200, 5-300, 5-500, 5-700, 5-1000, 20-100, 20-1000, 50-200, 50-500, 50-1000, or 50-100, bases long, including any range between. In another embodiment, the term “oligonucleotide” refers to a molecule comprising 5-100 bases. In another embodiment, the term “oligonucleotide” refers to a molecule comprising 5-80 bases. In another embodiment, the term “oligonucleotide” refers to a molecule comprising 5-40 bases. In another embodiment, the term “oligonucleotide” refers to a molecule comprising 50-100 bases. In another embodiment, the term “oligonucleotide” refers to a molecule comprising 20-70 bases. In another embodiment, the term “oligonucleotide” refers to a molecule comprising 5-30 bases. In another embodiment, the term “oligonucleotide” refers to a molecule comprising 5-25 bases. In another embodiment, the term “oligonucleotide” refers to a molecule comprising 10-50 bases, 20-50 bases, 5-50 bases, 10-100 bases, including any range between.

In some embodiments, the inhibitory nucleic acid is an RNA interfering molecule (RNAi). In some embodiments, the RNAi is or comprises double stranded RNA (dsRNA).

As used herein “an interfering RNA” refers to any double stranded or single stranded RNA sequence, capable-either directly or indirectly (i.e., upon conversion)—of inhibiting or down regulating gene expression by mediating RNA interference. Interfering RNA includes but is not limited to small interfering RNA (“siRNA”) and small hairpin RNA (“shRNA”). “RNA interference” refers to the selective degradation of a sequence-compatible messenger RNA transcript.

In some embodiments, the polynucleotide is chemically modified. In some embodiments, the chemical modification is a modification of a backbone of the polynucleotide. In some embodiments, the chemical modification is a modification of a sugar of the polynucleotide. In some embodiments, the chemical modification is a modification of a nucleobase of the polynucleotide. In some embodiments, the chemical modification increases stability of the polynucleotide in a cell. In some embodiments, the chemical modification increases stability of the polynucleotide in vivo. In some embodiments, the chemical modification increases the stability of the polynucleotide in vitro, such as, in the open air, field, on a surface exposed to air, etc. In some embodiments, the chemical modification increases the polynucleotide's ability to induce silencing of a target gene or sequence, including, but not limited to an RNA molecule derived from a pathogen or an RNA derived from a plant cell, as described herein. In some embodiments, the chemical modification is selected from: a phosphate-ribose backbone, a phosphate-deoxyribose backbone, a phosphorothioate-deoxyribose backbone, a 2′-O-methyl-phosphorothioate backbone, a phosphorodiamidate morpholino backbone, a peptide nucleic acid backbone, a 2-methoxyethyl phosphorothioate backbone, a constrained ethyl backbone, an alternating locked nucleic acid backbone, a phosphorothioate backbone, N3′-P5′ phosphoroamidates, 2′-deoxy-2′-fluoro-β-d-arabino nucleic acid, cyclohexene nucleic acid backbone nucleic acid, tricyclo-DNA (tcDNA) nucleic acid backbone, ligand-conjugated antisense, and a combination thereof.

### Methods

By another aspect, there is provided a method of treating a subject in need thereof, the method comprising administering to the subject a therapeutic composition of the invention.

According to another aspect, there is provided a method for delivering an active agent to a tissue or to a specific organ of a subject, the method comprising administering to the subject a therapeutically effective amount of the pharmaceutical composition of the invention described hereinabove, thereby delivering the active agent to the specific organ. In some embodiments, the specific organ is selected from heart, lungs, spleen, brain, kidney, or liver. In some embodiments, the tissue is a tumor tissue.

In some embodiments, the pharmaceutical composition is a heart targeting composition. In some embodiments, the pharmaceutical composition is for use in treating a heart disease, a heart disorder or a heart condition. In some embodiments, the pharmaceutical composition is a tumor targeting composition. In some embodiments, the pharmaceutical composition is for use in treating a tumor. In some embodiments, the pharmaceutical composition comprises a therapeutically effective amount of the active agent. The term “effective amount” refers to an amount effective, at a dosages and periods of time necessary to achieve a desired therapeutic result. It will be apparent to those of ordinary skill in the art that the therapeutically effective amount of the molecule according to the present invention will depend, inter alia upon the administration schedule, the unit dose of molecule administered, whether the molecule is administered in combination with other therapeutic agents, the immune status and health of the patient, the therapeutic activity of the molecule administered and the judgment of the treating physician.

By another aspect, there is provided a method of diagnosing a subject in need thereof, the method comprising administering to the subject a composition of the invention.

In some embodiments, the subject suffers from a disease. In some embodiments, the disease is treatable by the active agent. In some embodiments, the subject is at risk of a disease. In some embodiments, the subject is in need of determining if he/she has a disease. In some embodiments, the subject is in need of determining efficacy of an active agent. In some embodiments, the subject is in need of determining treatment. In some embodiments, determining treatment is determining with which active agent to treat. In some embodiments, determining treatment is determining the dose of active agent with which to treat. In some embodiments, determining treatment is determining the type of nanoparticle with which to treat.

In some embodiments, the method is a diagnostic method and the nanoparticle comprises an active agent and a nucleic acid molecule. In some embodiments, the method is a theranostic method and the nanoparticle comprises an active agent and a nucleic acid molecule. In some embodiments, the nucleic acid molecule uniquely identifies the active agent. In some embodiments, a nanoparticle comprises a particular active agent and a nucleic acid molecule with a particular sequence and the sequence and agent are known. In this way a skilled artisan can identify the active agent by the sequence as a different sequence is used for each different agent. In some embodiments, different agents are different doses of the same agent. In some embodiments, the nucleic acid molecule is a barcode.

In some embodiments, the method is a diagnostic method, and the nanoparticle comprises an active agent, wherein the active agent is a diagnostic agent.

In some embodiments, the method is a method of determining biodistribution. In some embodiments, biodistribution is biodistribution of the active agent. In some embodiments, biodistribution is biodistribution of the nanoparticle. In some embodiments, a plurality of types of nanoparticles are administered. In some embodiments, the barcodes of each type of nanoparticle uniquely identify the lipid composition of the nanoparticle. That is, various nanoparticles are generated with different lipid compositions and a specific barcode is loaded into each nanoparticle type such that a known barcode identifies each nanoparticle lipid composition. In some embodiments, the barcode sequence is a predetermined sequence. In some embodiments, the barcode sequence is a pre-known sequence. In some embodiments, biodistribution is determined by determining biodistribution of the barcode. In some embodiments, biodistribution is determined by the presence of the barcode. In some embodiments, the presence of a barcode/nucleic acid molecule in a given location in the subject is indicative of a nanoparticle having reached that given location. In some embodiments, a nanoparticle is the nanoparticle identified by the barcode/nucleic acid molecule. In some embodiments, a nanoparticle is the nanoparticle that contained the barcode/nucleic acid molecule. In some embodiments, a location is a tissue. In some embodiments, a location is a cell type. In some embodiments, a location is a disease site. In some embodiments, a location is a tumor. In some embodiments, the biodistribution is determined by the presence of the barcode/nucleic acid molecule.

In some embodiments, the method further comprises receiving a sample from the subject after the administering. In some embodiments, the method further comprises extracting a sample from the subject after the administering. In some embodiments, the sample is from the location. In some embodiments, the location is a plurality of locations. In some embodiments, the sample is from a location to be investigated for distribution. In some embodiments, the sample is tissue. In some embodiments, the sample is fluid. In some embodiments, the sample is a tumor. In some embodiments, the sample comprises disease cells.

In some embodiments, the method further comprises extracting nucleic acids from the sample. In some embodiments, the method further comprises purifying the nucleic acid molecule. Methods of nucleic acid extraction, isolation and purification are well known in the art and any such method may be employed. In some embodiments, the nucleic acid molecules are analyzed. In some embodiments, analyzed is analyzed for sequence. In some embodiments, the nucleic acid molecules are sequenced. In some embodiments, sequencing is deep sequencing. In some embodiments, sequencing is next generation sequencing.

### Definitions

As used herein, the term “alkyl” describes an aliphatic hydrocarbon including straight chain and branched chain groups. The term “alkyl”, as used herein, also encompasses saturated or unsaturated hydrocarbon, hence this term further encompasses alkenyl and alkynyl.

The term “alkenyl” describes an unsaturated alkyl, as defined herein, having at least two carbon atoms and at least one carbon-carbon double bond. The alkenyl may be substituted or unsubstituted by one or more substituents, as described hereinabove.

The term “alkynyl”, as defined herein, is an unsaturated alkyl having at least two carbon atoms and at least one carbon-carbon triple bond. The alkynyl may be substituted or unsubstituted by one or more substituents, as described hereinabove.

The term “cycloalkyl” describes an all-carbon monocyclic or fused ring (i.e. rings which share an adjacent pair of carbon atoms) group where one or more of the rings does not have a completely conjugated pi-electron system. The cycloalkyl group may be substituted or unsubstituted, as indicated herein.

The term “aryl” describes an all-carbon monocyclic or fused-ring polycyclic (i.e. rings which share adjacent pairs of carbon atoms) groups having a completely conjugated pi-electron system. The aryl group may be substituted or unsubstituted, as indicated herein.

The term “alkoxy” describes both an O-alkyl and an —O-cycloalkyl group, as defined herein. The term “aryloxy” describes an —O-aryl, as defined herein.

Each of the alkyl, cycloalkyl and aryl groups in the general formulas herein may be substituted by one or more substituents, whereby each substituent group can independently be, for example, halide, alkyl, alkoxy, cycloalkyl, nitro, amino, hydroxyl, thiol, thioalkoxy, carboxy, amide, aryl and aryloxy, depending on the substituted group and its position in the molecule. Additional substituents are also contemplated.

The term “halide”, “halogen” or “halo” describes fluorine, chlorine, bromine or iodine. The term “haloalkyl” describes an alkyl group as defined herein, further substituted by one or more halide(s). The term “haloalkoxy” describes an alkoxy group as defined herein, further substituted by one or more halide(s). The term “hydroxyl” or “hydroxy” describes a —OH group. The term “mercapto” or “thiol” describes a —SH group. The term “thioalkoxy” describes both an —S-alkyl group, and a —S-cycloalkyl group, as defined herein. The term “thioaryloxy” describes both an —S-aryl and a —S-heteroaryl group, as defined herein. The term “amino” describes a —NR′R″ group, or a salt thereof, with R′ and R″ as described herein.

The term “heterocyclyl” describes a monocyclic or fused ring group having in the ring(s) one or more atoms such as nitrogen, oxygen and sulfur. The rings may also have one or more double bonds. However, the rings do not have a completely conjugated pi-electron system. Representative examples are piperidine, piperazine, tetrahydrofuran, tetrahydropyran, morpholino and the like.

The term “carboxy” describes a —C(O)OR′ group, or a carboxylate salt thereof, where R′ is hydrogen, alkyl, cycloalkyl, alkenyl, aryl, heteroaryl (bonded through a ring carbon) or heterocyclyl (bonded through a ring carbon) as defined herein. or “carboxylate”.

The term “carbonyl” describes a —C(O)R′ group, where R′ is as defined hereinabove. The above-terms also encompass thio-derivatives thereof (thiocarboxy and thiocarbonyl).

The term “thiocarbonyl” describes a —C(S)R′ group, where R′ is as defined hereinabove. A “thiocarboxy” group describes a —C(S)OR′ group, where R′ is as defined herein. A “sulfinyl” group describes an —S(O)R′ group, where R′ is as defined herein. A “sulfonyl” or “sulfonate” group describes an —S(O)2R′ group, where R′ is as defined herein.

A “carbamyl” or “carbamate” group describes an —OC(O)NR′R″ group, where R′ is as defined herein and R″ is hydrogen, alkyl, cycloalkyl, alkenyl, aryl, heteroaryl (bonded through a ring carbon) or heterocyclyl (bonded through a ring carbon) as defined herein. A “nitro” group refers to a —NO₂ group. The term “amide” as used herein encompasses C-amide and N-amide. The term “C-amide” describes a —C(O)NR′R″ end group or a —C(O)NR′-linking group, as these phrases are defined hereinabove, where R′ and R″ are as defined herein. The term “N-amide” describes a —NR″C(O)R′ end group or a —NR′C(O)-linking group, as these phrases are defined hereinabove, where R′ and R″ are as defined herein.

A “cyano” or “nitrile” group refers to a —CN group. The term “azo” or “diazo” describes an —N═NR′ end group or an —N═N-linking group, as these phrases are defined hereinabove, with R′ as defined hereinabove. The term “guanidine” describes a —R′NC(N)NR″R′″ end group or a —R′NC(N)NR″-linking group, as these phrases are defined hereinabove, where R′, R″ and R″ are as defined herein. As used herein, the term “azide” refers to a —N3 group. The term “sulfonamide” refers to a —S(O)2NR′R″ group, with R′ and R″ as defined herein.

The term “phosphonyl” or “phosphonate” describes an —OP(O)—(OR′)2 group, with R′ as defined hereinabove. The term “phosphinyl” describes a —PR′R″ group, with R′ and R″ as defined hereinabove. The term “alkylaryl” describes an alkyl, as defined herein, which is substituted by an aryl, as described herein. An exemplary alkylaryl is benzyl.

The term “heteroaryl” describes a monocyclic or fused ring (i.e. rings which share an adjacent pair of atoms) group having in the ring(s) one or more atoms, such as, for example, nitrogen, oxygen and sulfur and, in addition, having a completely conjugated pi-electron system. As used herein, the term “heteroaryl” refers to an aromatic ring in which at least one atom forming the aromatic ring is a heteroatom. Heteroaryl rings can be formed by three, four, five, six, seven, eight, nine and more than nine atoms. Heteroaryl groups can be optionally substituted. Examples of heteroaryl groups include, but are not limited to, aromatic C3-8 heterocyclic groups containing one oxygen or sulfur atom, or two oxygen atoms, or two sulfur atoms or up to four nitrogen atoms, or a combination of one oxygen or sulfur atom and up to two nitrogen atoms, and their substituted as well as benzo- and pyrido-fused derivatives, for example, connected via one of the ring-forming carbon atoms. In certain embodiments, heteroaryl is selected from among oxazolyl, isoxazolyl, oxadiazolyl, thiazolyl, isothiazolyl, pyridinyl, pyridazinyl, pyrimidinal, pyrazinyl, indolyl, benzimidazolyl, quinolinyl, isoquinolinyl, quinazolinyl or quinoxalinyl.

In some embodiments, a heteroaryl group is selected from among pyrrolyl, furanyl (furyl), thiophenyl (thienyl), imidazolyl, pyrazolyl, 1,2,3-triazolyl, 1,2,4-triazolyl, 1,3-oxazolyl (oxazolyl), 1,2-oxazolyl (isoxazolyl), oxadiazolyl, 1,3-thiazolyl (thiazolyl), 1,2-thiazolyl (isothiazolyl), tetrazolyl, pyridinyl (pyridyl)pyridazinyl, pyrimidinyl, pyrazinyl, 1,2,3-triazinyl, 1,2,4-triazinyl, 1,3,5-triazinyl, 1,2,4,5-tetrazinyl, indazolyl, indolyl, benzothiophenyl, benzofuranyl, benzothiazolyl, benzimidazolyl, benzodioxolyl, acridinyl, quinolinyl, isoquinolinyl, quinazolinyl, quinoxalinyl, phthalazinyl, thienothiophenyl, 1,8-naphthyridinyl, other naphthyridinyls, pteridinyl or phenothiazinyl. Where the heteroaryl group includes more than one ring, each additional ring is the saturated form (perhydro form) or the partially unsaturated form (e.g., the dihydro form or tetrahydro form) or the maximally unsaturated (nonaromatic) form. The term heteroaryl thus includes bicyclic radicals in which the two rings are aromatic and bicyclic radicals in which only one ring is aromatic. Such examples of heteroaryl include 3H-indolinyl, 2 (1H)-quinolinonyl, 4-oxo-1,4-dihydroquinolinyl, 2H-1-oxoisoquinolyl, 1,2-dihydroquinolinyl, (2H) quinolinyl N-oxide, 3,4-dihydroquinolinyl, 1,2-dihydroisoquinolinyl, 3,4-dihydro-isoquinolinyl, chromonyl, 3,4-dihydroiso-quinoxalinyl, 4-(3H) quinazolinonyl, 4H-chromenyl, 4-chromanonyl, oxindolyl, 1,2,3,4-tetrahydroisoquinolinyl, 1,2,3,4-tetrahydro-quinolinyl, 1H-2,3-dihydroisoindolyl, 2,3-dihydrobenzo[f]isoindolyl, 1,2,3,4-tetrahydrobenzo-[g]isoquinolinyl, 1,2,3,4-tetrahydro-benzo[g]isoquinolinyl, chromanyl, isochromanonyl, 2,3-dihydrochromonyl, 1,4-benzo-dioxanyl, 1,2,3,4-tetrahydro-quinoxalinyl, 5,6-dihydro-quinolyl, 5,6-dihydroiso-quinolyl, 5,6-dihydroquinoxalinyl, 5,6-dihydroquinazolinyl, 4,5-dihydro-1H-benzimidazolyl, 4,5-dihydro-benzoxazolyl, 1,4-naphthoquinolyl, 5,6,7,8-tetrahydro-quinolinyl, 5,6,7,8-tetrahydro-isoquinolyl, 5,6,7,8-tetrahydroquinoxalinyl, 5,6,7,8-tetrahydroquinazolyl, 4,5,6,7-tetrahydro-1H-benzimidazolyl, 4,5,6,7-tetrahydro-benzoxazolyl, 1H-4-oxa-1,5-diaza-naphthalen-2-onyl, 1,3-dihydroimidizolo-[4,5]-pyridin-2-onyl, 2,3-dihydro-1,4-dinaphtho-quinonyl, 2,3-dihydro-1H-pyrrol[3,4-b]quinolinyl, 1,2,3,4-tetrahydrobenzo[b]-[1,7]naphthyridinyl, 1,2,3,4-tetra-hydrobenz[b][1,6]-naphthyridinyl, 1,2,3,4-tetrahydro-9H-pyrido[3,4-b]indolyl, 1,2,3,4-tetrahydro-9H-pyrido[4,3-b]indolyl, 2,3-dihydro-1H-pyrrolo-[3,4-b]indolyl, 1H-2,3,4,5-tetrahydro-azepino[3,4-b]indolyl, 1H-2,3,4,5-tetrahydroazepino-[4,3-b]indolyl, 1H-2,3,4,5-tetrahydro-azepino[4,5-b]indolyl, 5,6,7,8-tetrahydro[1,7]napthyridinyl, 1,2,3,4-tetrahydro-[2,7]-naphthyridyl, 2,3-dihydro[1,4]dioxino[2,3-b]pyridyl, 2,3-dihydro[1,4]-dioxino[2,3-b]pryidyl, 3,4-dihydro-2H-1-oxa[4,6]diazanaphthalenyl, 4,5,6,7-tetrahydro-3H-imidazo-[4,5-c]pyridyl, 6,7-dihydro[5,8]diazanaphthalenyl, 1,2,3,4-tetrahydro[1,5]-napthyridinyl, 1,2,3,4-tetrahydro[1,6]napthyridinyl, 1,2,3,4-tetrahydro[1,7]napthyridinyl, 1,2,3,4-tetrahydro-[1,8]napthyridinyl or 1,2,3,4-tetrahydro[2,6]napthyridinyl. In some embodiments, heteroaryl groups are optionally substituted. In one embodiment, the one or more substituents are each independently selected from among halo, hydroxy, amino, cyano, nitro, alkylamido, acyl, C1-6-alkyl, C1-6-haloalkyl, C1-6-hydroxyalkyl, C1-6-aminoalkyl, C1-6-alkylamino, alkylsulfenyl, alkylsulfinyl, alkylsulfonyl, sulfamoyl, or trifluoromethyl.

Examples of heteroaryl groups include, but are not limited to, unsubstituted and mono- or di-substituted derivatives of furan, benzofuran, thiophene, benzothiophene, pyrrole, pyridine, indole, oxazole, benzoxazole, isoxazole, benzisoxazole, thiazole, benzothiazole, isothiazole, imidazole, benzimidazole, pyrazole, indazole, tetrazole, quinoline, isoquinoline, pyridazine, pyrimidine, purine and pyrazine, furazan, 1,2,3-oxadiazole, 1,2,3-thiadiazole, 1,2,4-thiadiazole, triazole, benzotriazole, pteridine, phenoxazole, oxadiazole, benzopyrazole, quinolizine, cinnoline, phthalazine, quinazoline and quinoxaline. In some embodiments, the substituents are halo, hydroxy, cyano, O—C₁₋₆-alkyl, C₁₋₆-alkyl, hydroxy-C₁₋₆-alkyl and amino-C₁₋₆-alkyl.

As used herein, the terms “halo” and “halide”, which are referred to herein interchangeably, describe an atom of a halogen, that is fluorine, chlorine, bromine or iodine, also referred to herein as fluoride, chloride, bromide and iodide.

### General

As used herein, the term “about” when combined with a value refers to plus and minus 10% of the reference value. For example, a length of about 1000 nanometers (nm) refers to a length of 1000 nm+−100 nm.

It is noted that as used herein and in the appended claims, the singular forms “a,” “an,” and “the” include plural referents unless the context clearly dictates otherwise. Thus, for example, reference to “a polynucleotide” includes a plurality of such polynucleotides and reference to “the polypeptide” includes reference to one or more polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements or use of a “negative” limitation.

In those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

Various embodiments and aspects of the present invention as delineated hereinabove and as claimed in the claims section below find experimental support in the following examples.

### EXAMPLES

Generally, the nomenclature used herein, and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, CT (1994); Mishell and Shiigi (eds), “Strategies for Protein Purification and Characterization—A Laboratory Course Manual” CSHL Press (1996); all of which are incorporated by reference. Other general references are provided throughout this document.

### Example 1

Exemplary compounds of the invention have been synthesized according to synthetic schemes presented in Example 3. The following compounds have been synthesized:

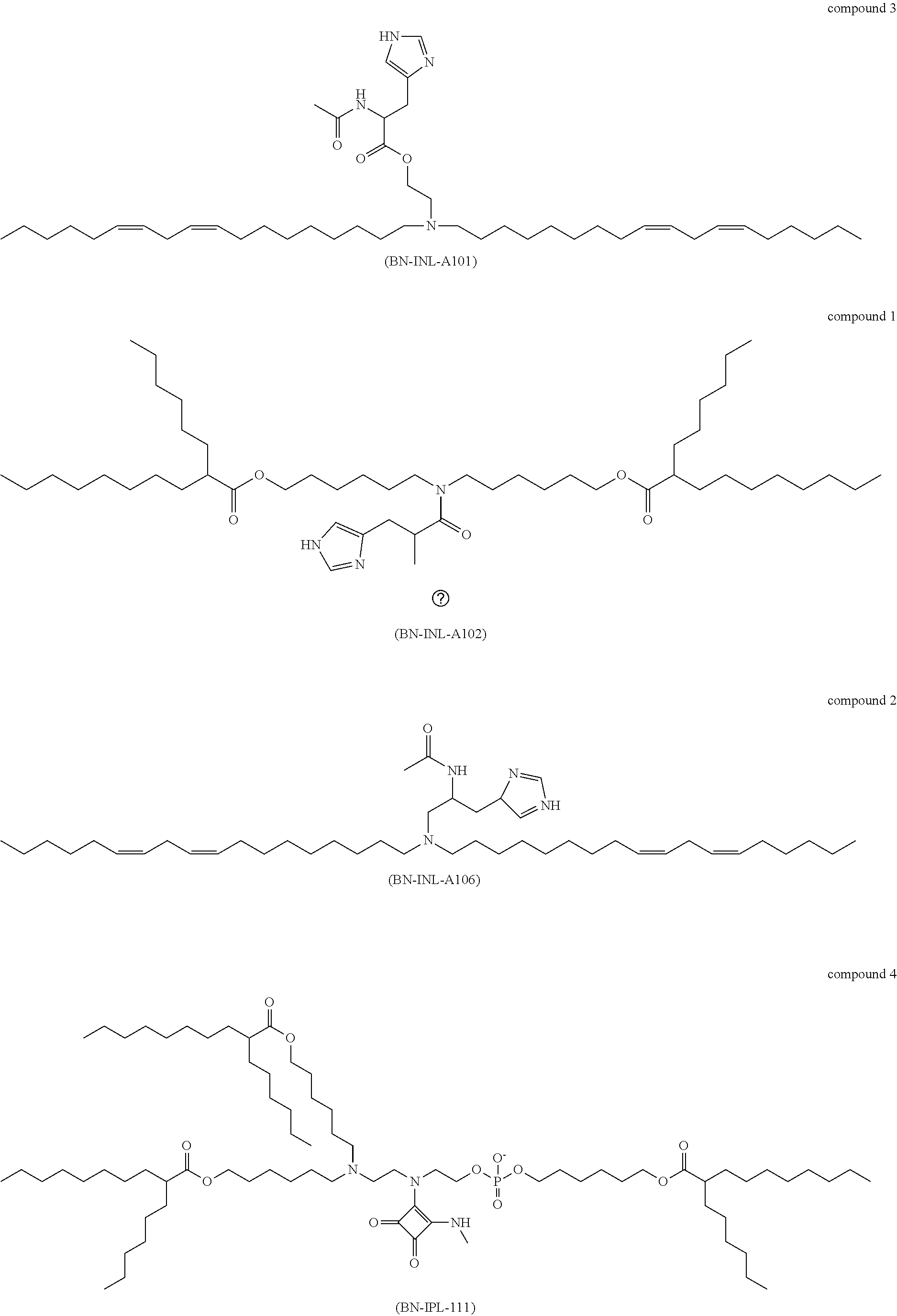

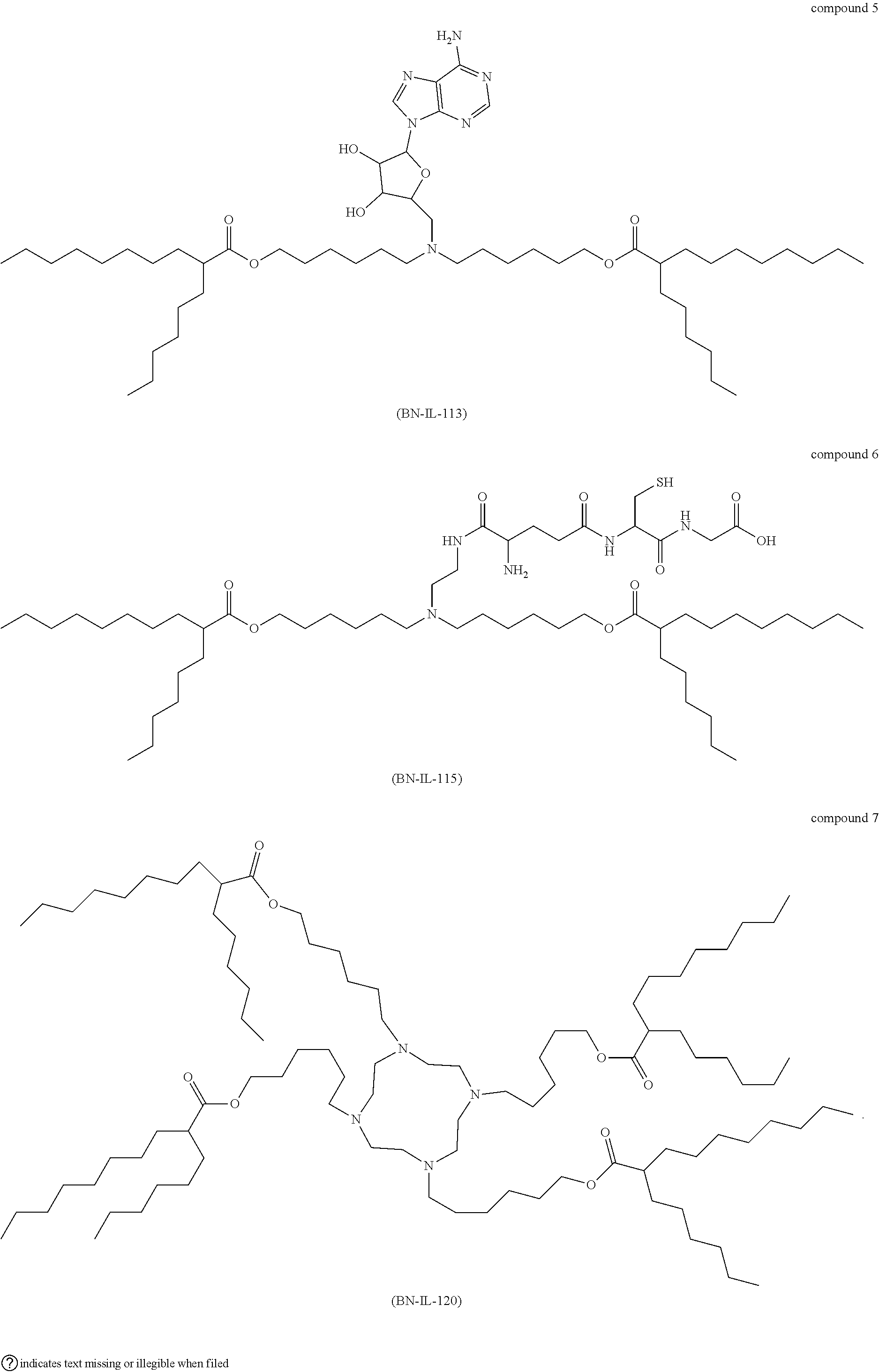

Additional compounds listed herein can be synthesized based on the synthetic schemes presented herein. Other possible synthetic strategies are well-known to a skilled artisan.

The inventors successfully utilized the abovementioned exemplary compounds of the invention for the preparation of stable LNPs.

A composition of an exemplary LNP is as follows:
phospholipid (e.g., DSPC and/or DOPE) about 5-15 mol %; sterol (e.g., cholesterol) about 30-45 mol %; compound of the invention (i.e. BN-INL-A101/A102) about 40-50 mol %, and optionally PEG-Lipid (e.g. DSPE-PEG2000). The LNPs prepared by the inventors have been characterized by an average particle size ranging between about 60 and about 120 nm.

For the preparation of the LNPs, there are two phases: an organic and an aqueous phase. The organic phase contains the lipids (including the liposome-, and optionally non-liposome forming lipids and the compound of the invention) and the aqueous phase contains the active agent (e.g., a polynucleic acid). These phases are rapidly mixed together at a low pH via microfluidics. The solution is brought to neutral pH to obtain LNPs with relatively neutral surface charge. The LNPs are characterized by measuring the size and PDI using dynamic light scattering and zeta potential using electrophoretic mobility. The compositions of the LNPs are checked via quantitative HPLC. Here it is possible to test the stability of the lipids within the LNP by looking for novel peaks corresponding to breakdown products. It is also possible to test the LNPs stability over time using the HPLC method. The encapsulation efficiency is also calculated by measuring the ratio of polynucleic acid contained in the LNP to the total nucleic acid concentration via a fluorescence assay. The total encapsulated content of the polynucleic acid has been determined by measuring total content vs. free content of the polynucleic acid.

The inventors determined biodistribution of the exemplary LNPs. The LNPs have been injected into mice. The delivered nucleic acids from various organs of these mice have been extracted and the copy number has been quantified by digital PCR. The normalized copy numbers have been compared to determine biodistribution of a given LNP.

### Study 1

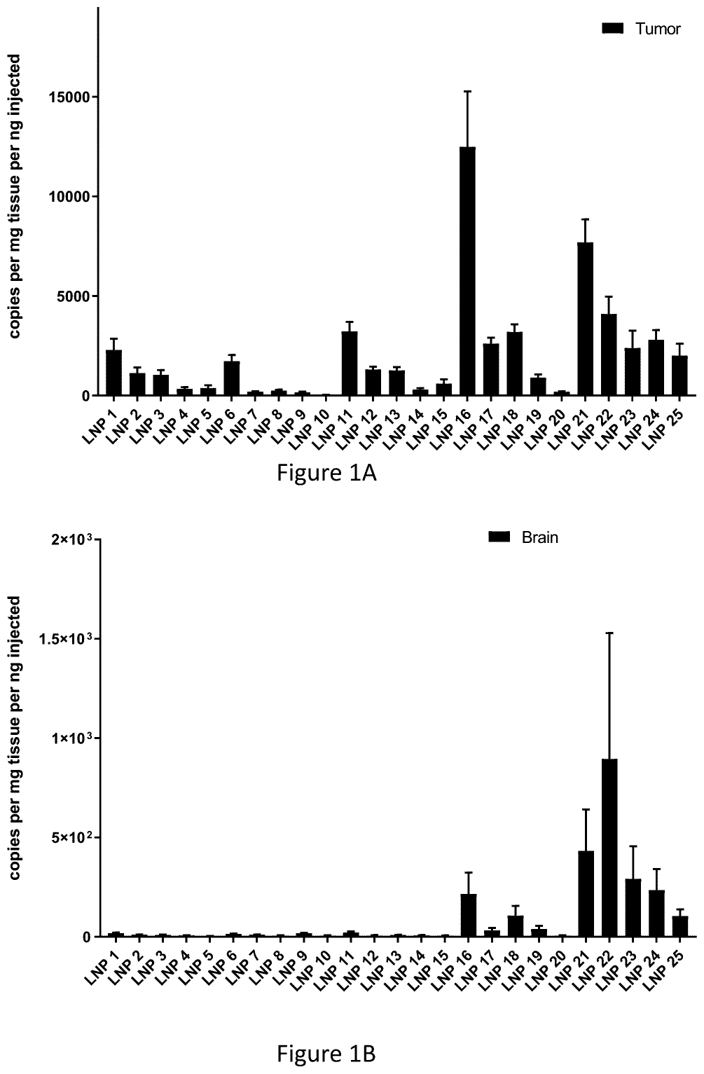

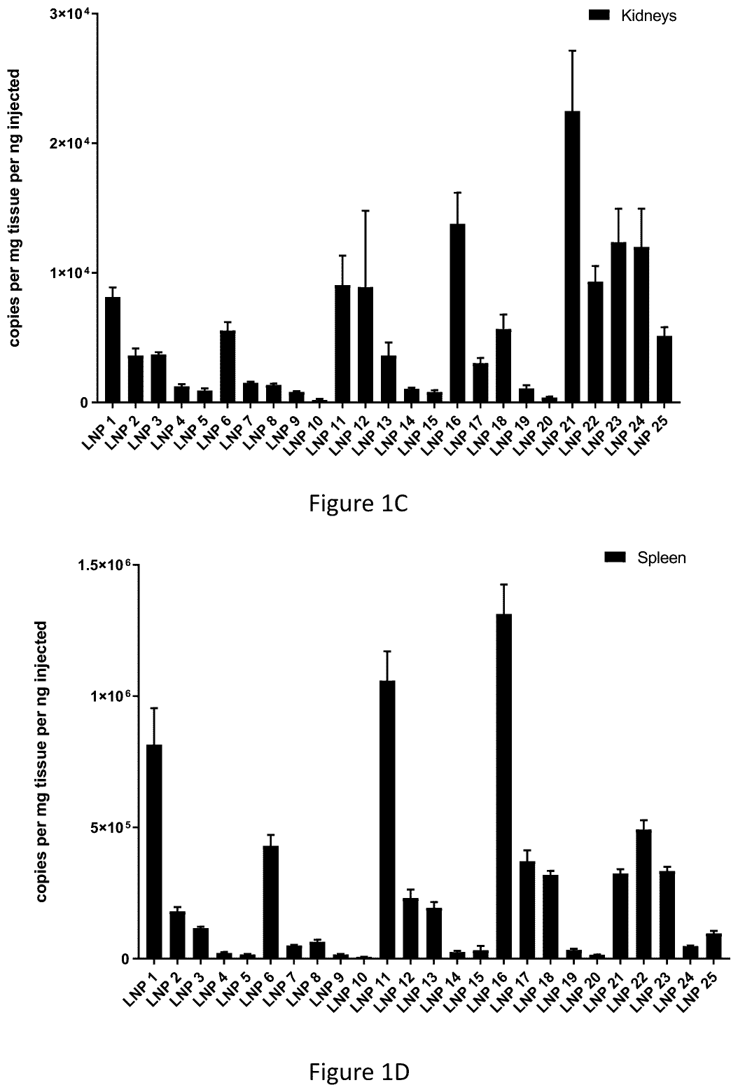

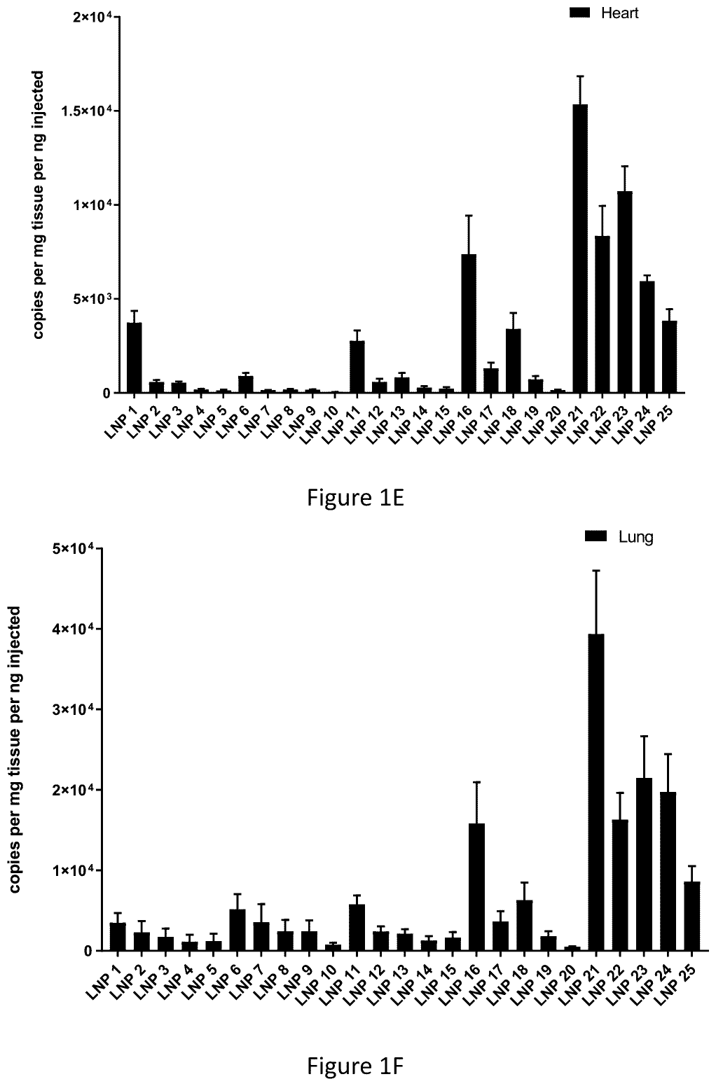

More specifically, BAlb/c female mice were injected with murine breast cancer 4T1-Luc cells. 10-14 days post injection, animals (n=4) were injected with different mixtures of LNP: group 1 with LNPs 1-5, group 2 with LNPs 6-10, group 3 with LNPs 10-15, group 4 with LNPs 16-20 and group 5 with LNPs 20-25. 4-6 hours later, blood samples were collected before animals were perfused and sacrificed. Organs were collected (Brain, Heart, Lungs, Liver, Kidneys, Thymus, Spleen, Ovaries and tumor) and immediately frozen in liquid nitrogen. Barcodes were extracted from each tissue and quantified by droplet digital PCR. Results are expressed as barcode copies per mg tissue, normalized to barcode injected dose. Results for each LNP were compared to results obtained for LNP1 which is similar to Onpattro, an FDA approved LNP in clinical use. The results of these experiments are presented in FIGS. 1A-1E.

The chemical compositions of the tested LNPs are as follows:

| | | | Molar Composition (%) | | | | | Physical Characterization | |
|---|---|---|---|---|---|---|---|---|---|
| LNP | Ion- | Helper | Ion- | Helper | | DMG- | Ionizable | Average | Zeta potential |
| ID | Lipid | Lipid | lipid | Lipid | Cholesterol | PEG₂₀₀₀ | N:P ratio | size (nm) | (mV) |
| 1 | MC3 | DSPC | 50 | 10 | 38.5 | 1.5 | 6 | 118 | −3.1 |
| 2 | MC3 | DOPE | 50 | 10 | 38.5 | 1.5 | 6 | 99 | −3.2 |
| 3 | MC3 | DSPC + | 50 | 5/5 | 38.5 | 1.5 | 6 | 92 | −2.9 |
| | | DOPE | | | | | | | |
| 4 | MC3 | DSPC | 40 | 15 | 42 | 3 | 6 | 73 | −2.9 |
| 5 | MC3 | DOPE | 40 | 15 | 42 | 3 | 6 | 62 | −1.6 |
| 6 | DODAP | DSPC | 50 | 10 | 38.5 | 1.5 | 6 | 75 | −9.1 |
| 7 | DODAP | DOPE | 50 | 10 | 38.5 | 1.5 | 6 | 85 | −8.8 |
| 8 | DODAP | DSPC + | 50 | 5/5 | 38.5 | 1.5 | 6 | 81 | −10.2 |
| | | DOPE | | | | | | | |
| 9 | DODAP | DSPC | 40 | 15 | 42 | 3 | 6 | 68 | −10.0 |
| 10 | DODAP | DOPE | 40 | 15 | 42 | 3 | 6 | 57 | −7.6 |
| 11 | MC3 | DSPC | 50 | 10 | 38.5 | 1.5 | 4 | 103 | −2.0 |
| 12 | MC3 | DOPE | 50 | 10 | 38.5 | 1.5 | 4 | 106 | −2.6 |
| 13 | MC3 | DSPC + | 50 | 5/5 | 38.5 | 1.5 | 4 | 100 | −2.5 |
| | | DOPE | | | | | | | |
| 14 | MC3 | DSPC | 40 | 15 | 42 | 3 | 4 | 69 | −5.1 |
| 15 | MC3 | DOPE | 40 | 15 | 42 | 3 | 4 | 61 | −3.7 |
| 16 | BN-INL- | DSPC | 50 | 10 | 38.5 | 1.5 | 6 | 114 | −0.7 |
| | A101 | | | | | | | | |
| 17 | BN-INL- | DOPE | 50 | 10 | 38.5 | 1.5 | 6 | 85 | −4.1 |
| | A101 | | | | | | | | |
| 18 | BN-INL- | DSPC + | 50 | 5/5 | 38.5 | 1.5 | 6 | 87 | −2.2 |
| | A101 | DOPE | | | | | | | |
| 19 | BN-INL- | DSPC | 40 | 15 | 42 | 3 | 6 | 88 | −2.8 |
| | A101 | | | | | | | | |
| 20 | BN-INL- | DOPE | 40 | 15 | 42 | 3 | 6 | 61 | −2.1 |
| | A101 | | | | | | | | |
| 21 | BN-INL- | DSPC | 50 | 10 | 38.5 | 1.5 | 6 | 96 | −5.5 |
| | A102 | | | | | | | | |
| 22 | BN-INL- | DOPE | 50 | 10 | 38.5 | 1.5 | 6 | 111 | −6.1 |
| | A102 | | | | | | | | |
| 23 | BN-INL- | DSPC + | 50 | 5/5 | 38.5 | 1.5 | 6 | 95 | −8.0 |
| | A102 | DOPE | | | | | | | |
| 24 | BN-INL- | DSPC | 40 | 15 | 42 | 3 | 6 | 94 | −5.5 |
| | A102 | | | | | | | | |
| 25 | BN-INL- | DOPE | 40 | 15 | 42 | 3 | 6 | 75 | −5.8 |
| | A102 | | | | | | | | |

Based on the biodistribution analysis using the proprietary barcoded platform of the inventors, formulations containing BN-INL-A101 or BN-INL-A102 lipids systematically improved delivery to tested tissues by at least one order of magnitude. Improvement was relative to formulation similar in composition to FDA approved LNP.

### Example 2

The inventors have successfully manufactured stable LNPs, according to the method disclosed above. The composition of the stable LNPs was as follows: compound of the invention (BN-INL-A101, BN-INL-A102, BN-INL-A106, BN-INL-A113, BN-INL-A115, and BN-INL-A120, the structures of which are depicted above) between 30% and 60 mol %, helper lipid (e.g., BN-IPL-A111, and/or any of: DOPE, DOPC, and DSPC) between 3% and 25 mol %, PEG-lipid (e.g. DMG-PEG2000, PEG2000-C-DMG, or PEG2000-DSPE) between 0.5% and about 4.5 mol %, cholesterol between 25% and about 45 mol %, and a N:P ratio of between 3 and 12.

Furthermore, the inventors successfully prepared and tested LNPs encapsulating RNA, by implementing the following compounds of the invention (as ionizable lipids): BN-INL-A102, BN-INL-A106, and BN-INL-A113.

### Study 2

Mice (n=6) were injected with 83 different LNPs compositions, group 1 with 50 LNPs per mouse, group 2 with 34 LNPs per mouse. 4-6 hours following item injection, barcodes were quantified in different collected organs as previously described.

Formulation Ranges for injected particles: ionizable lipid 30% to 60%, helper lipid 5% to 25%, PEG 0.5% to 4.5%, cholesterol 25.5% to 44.5% with a N:P ratio of between 3 and 12. The resulting LNPs have been characterized by PDI≤0.3, average nanoparticle size≤250 nm and encapsulated material (barcode) concentration ≥21 ng/ul and are thus suitable for injection to mice. pKa of formulations has also been assessed and ranges from 4.3 to 9.2.

Biodistribution of the different formulations was analyzed in a large panel of tissues 4-6 hours following injection of the particles.

Biodistribution of the tested LNPs was analyzed. Some of the tested LNP showed enhanced accumulation within a specific organ (or within tumor), as compared to control. Based on the analysis of the biodistribution results, the inventors have identified specific ranges of LNP constituents which contribute to increased affinity of the LNPs of the invention to specific organs, as compared to the control (D-Lin-MC3-DMA lipid based LNP formulation). Table 1 summarizes the specific ranges of various LNP constituents.

TABLE 1
LNPs targeted formulations

| | Tumor | Heart | Liver | Spleen | Kidney | Lung |
|---|---|---|---|---|---|---|
| Ionizable | 35 to 55 | 35 to 60 | 30 to 60 | 35 to 50 | 35 to 55 | 35 to 60 |
| lipid [mol %] | | | | | | |
| Helper | 5 to 25 | 10 to 20 | 5 to 25 | 5 to 25 | 10 to 25 | 5 to 25 |
| lipid[mol %] | | | | | | |
| PEG lipid | 1.5 to 4.5 | 0.5 to 4.5 | 0.5 to 4.5 | 0.5 to 4.5 | 1.5 to 4.5 | 0.5 to 4.5 |
| [mol %] | | | | | | |
| Cholesterol | 25.5 to 43.5 | 27.5 to 44.5 | 25.5 to 44.5 | 35.5 to 44.5 | 30.5 to 38.5 | 25.5 to 44.5 |
| [mol %] | | | | | | |
| N:P ratio | 3 to 12 | 3 to 12 | 3 to 12 | 3 to 9 | 3 to 9 | 3 to 12 |

Some of the LNPs showed superior affinity towards specific organs. Exemplary LNPs with enhanced organ (or tumor) affinity/specificity along with organ accumulation of the LNPs vers. control LNP are summarized in Table 2 below.

TABLE 2
Exemplary LNPs with improved specificity

| | Tumor | Heart | Liver | Spleen | Brain | Lung |
|---|---|---|---|---|---|---|
| Ionizable | 50 mol % | 50 mol % | 50 mol % | 50 mol % | 55 mol % | 35 mol % |
| lipid | (BN-INL- | (BN-INL- | (BN-INL- | (BN-INL- | BN-INL- | BN-INL- |
| | A113) | A106) | A120) | A120) | A113 | A102 |
| Helper lipid | 10 mol % | 10 mol % | 5 mol % | 10 mol % | 10 mol % | 25 mol % |
| | (DSPC) | (DOPC) | (DOPC) | (DOPC) | (DSPC) | (DSPC) |
| PEG lipid | 1.5 mol % | 1.5 mol % | 4.5mol % | 1.5 mol % | 4.5 mol % | 1.5 mol % |
| | (PEG2000- | (PEG2000- | (PEG2000- | (PEG2000- | (PEG2000- | (PEG2000- |
| | DSPE) | DSPE) | DSPE) | DSPE) | DSPE) | DSPE) |
| Cholesterol | 38.5 mol % | 38.5 mol % | 40.5 mol % | 38.5 mol % | 30.5 mol % | 38.5 mol % |
| N:P ratio | 4 | 4 | 12 | 4 | 3 | 9 |
| % | 10 | 13 | 44 | 8 | 0.02 | 19 |
| accumulation | | | | | | |
| within a | | | | | | |
| specific | | | | | | |
| organ | | | | | | |
| (Control) | | | | | | |
| % | 38 | 68 | 94 | 15.5 | 0.3 | 27.3 |
| accumulation | | | | | | |
| within a | | | | | | |
| specific | | | | | | |
| organ (LNP | | | | | | |
| of the | | | | | | |
| invention) | | | | | | |

### Study 3

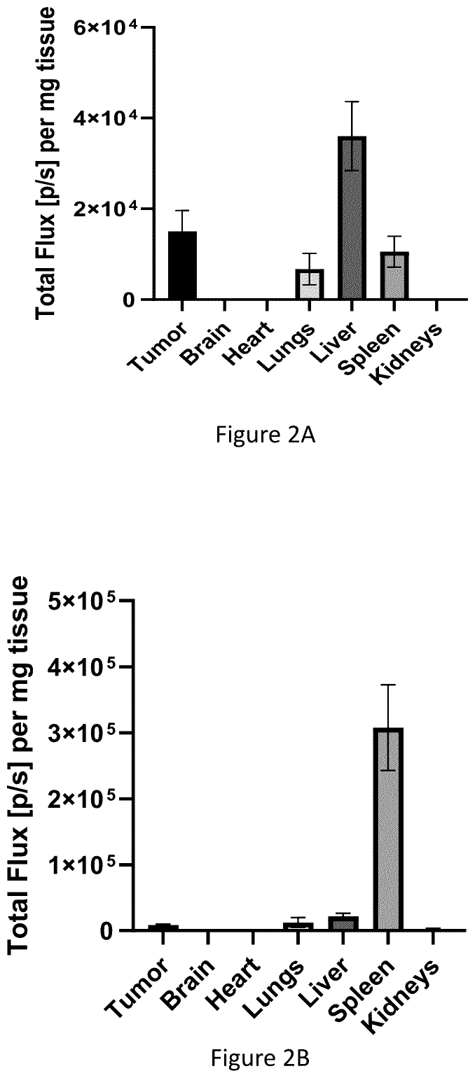

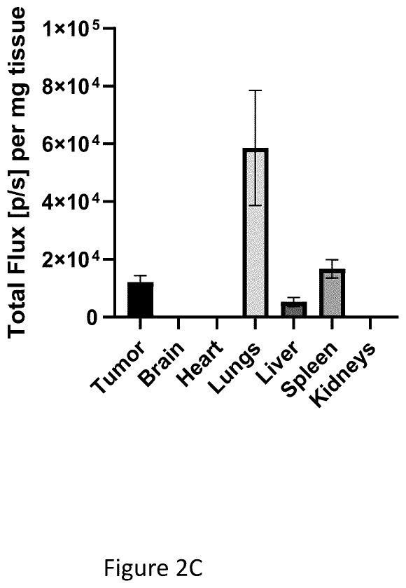

To prove the efficiency of the LNPs of the invention, LNPs based on formulations from study 2 were encapsulated with barcode and the luciferase mRNA. The activity of luciferase (by determining the generated luminescence) was recorded under live imaging. Results are summarized in FIGS. 2A-2C.

### Example 3

Exemplary compounds of the invention have been synthesized according to synthetic schemes presented hereinbelow.

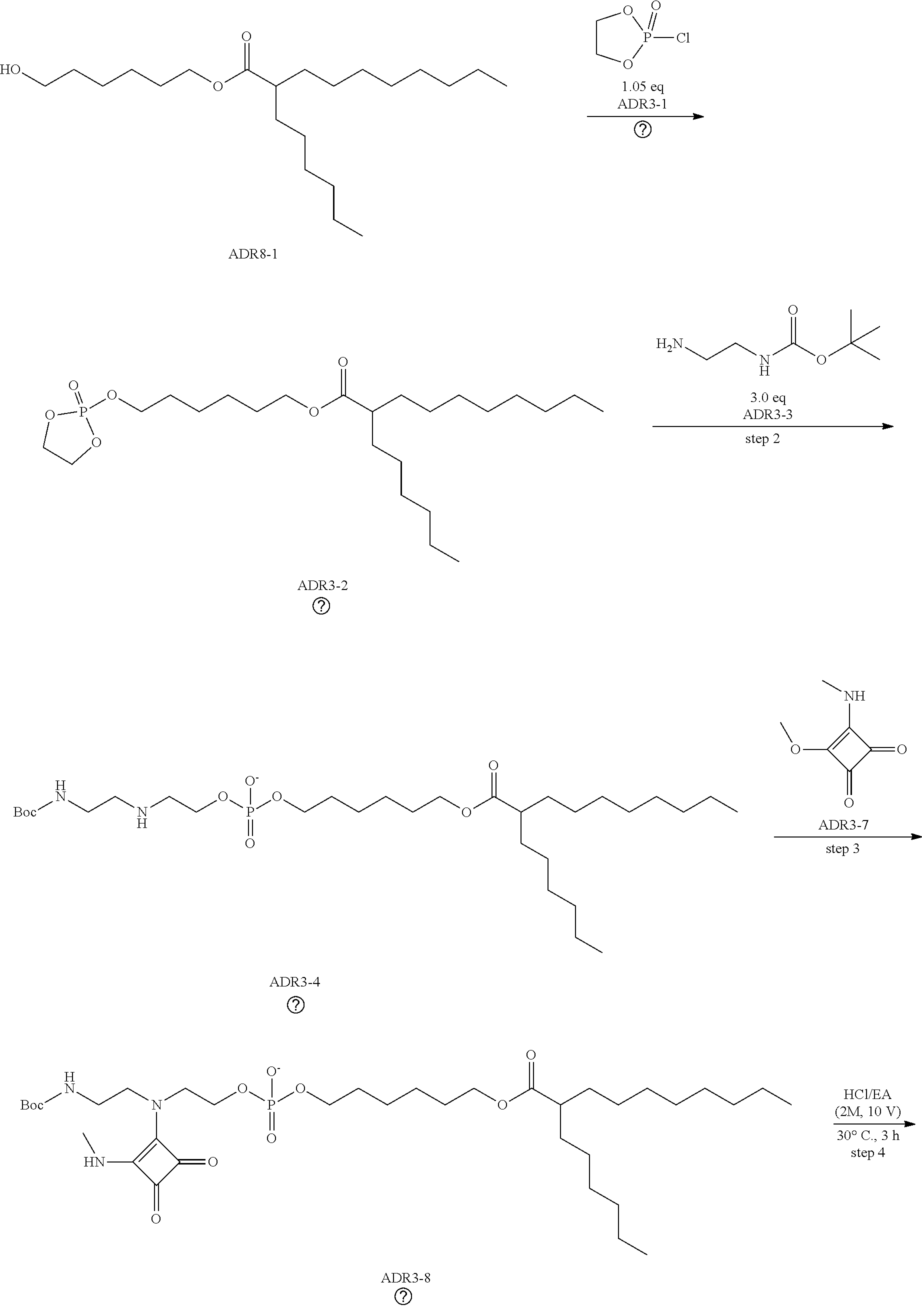

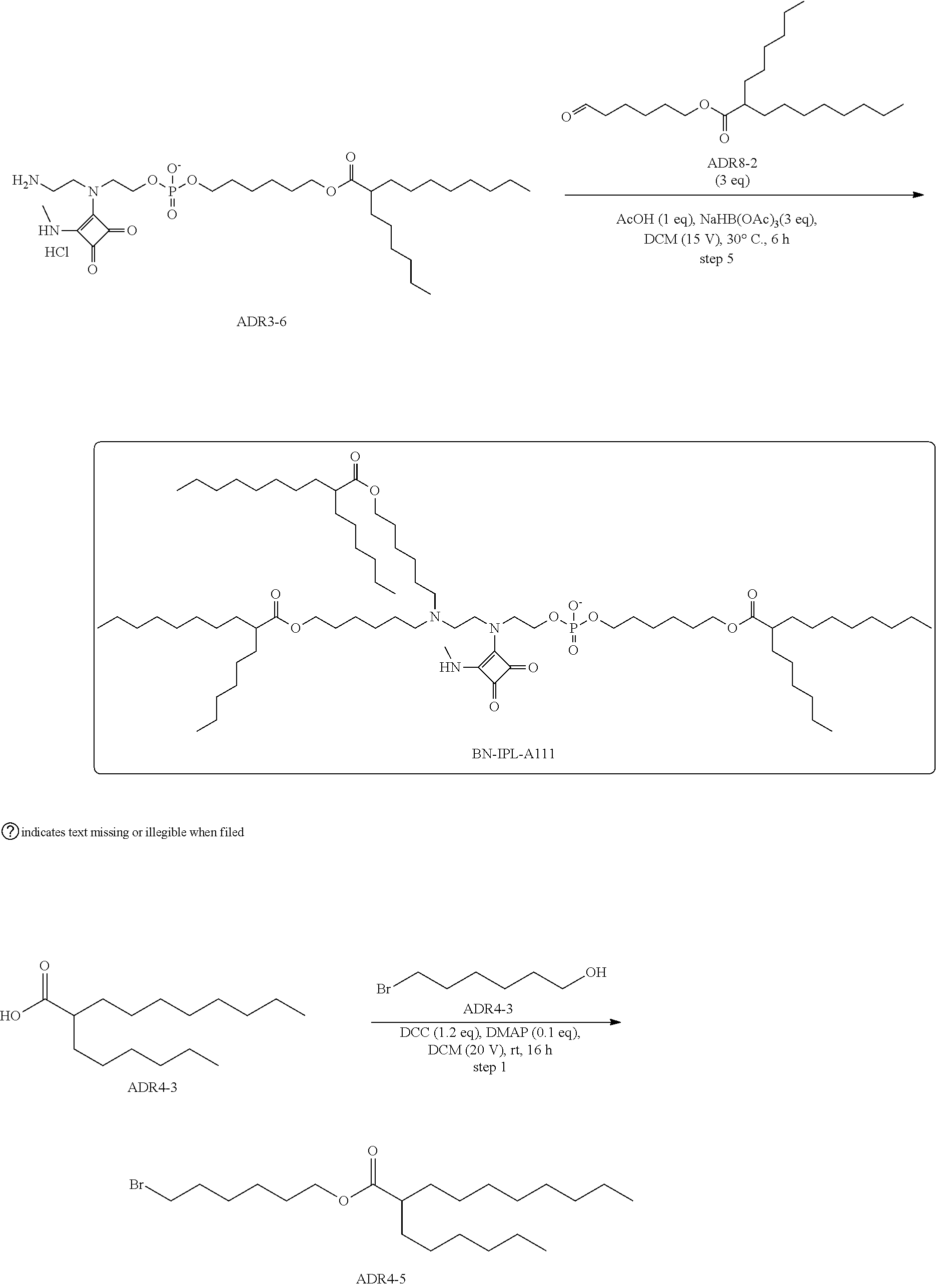

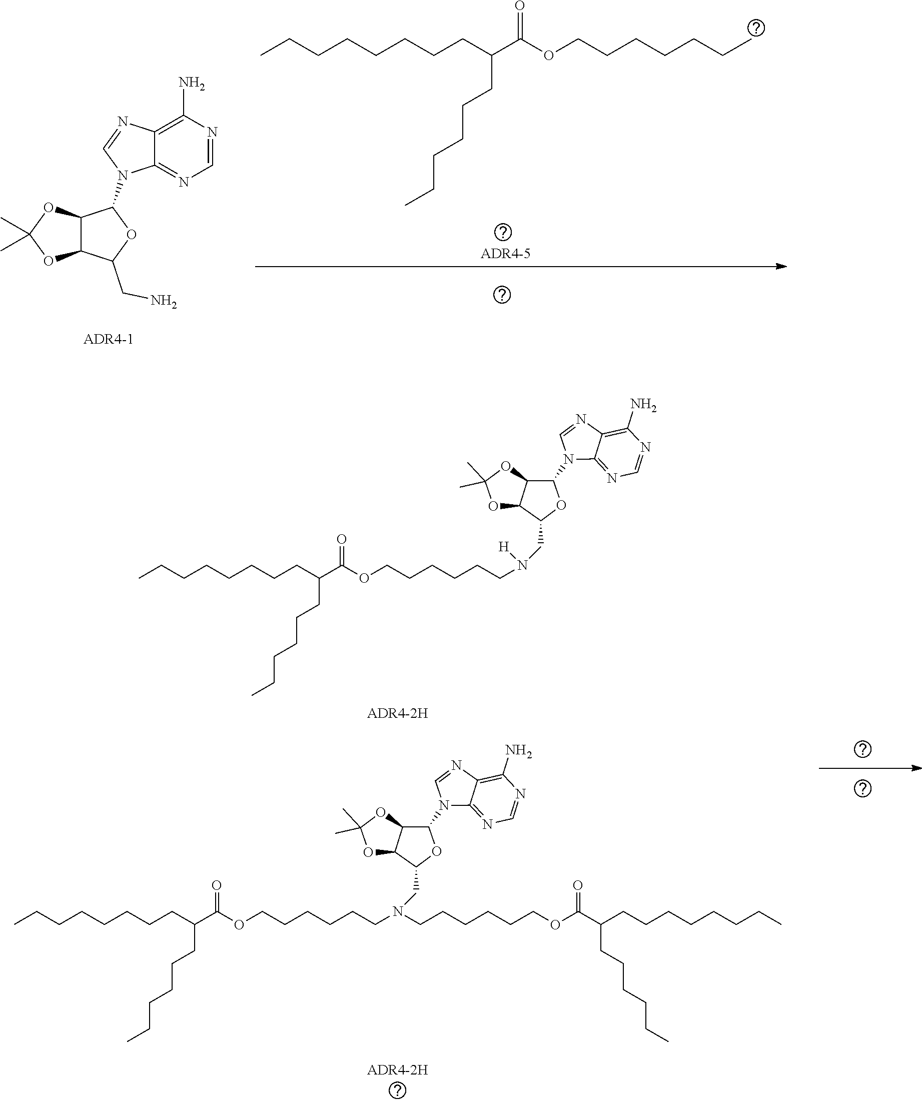

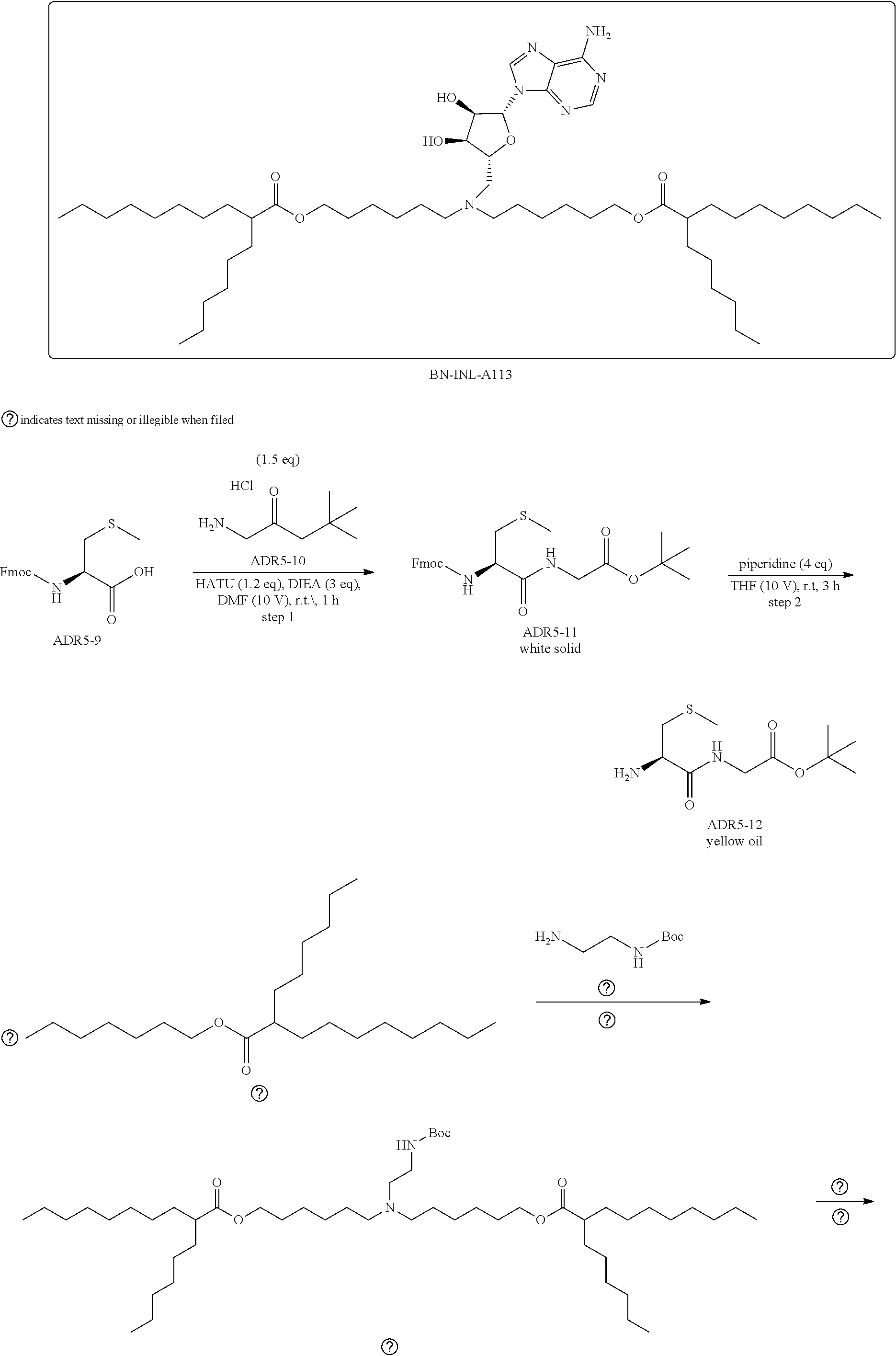

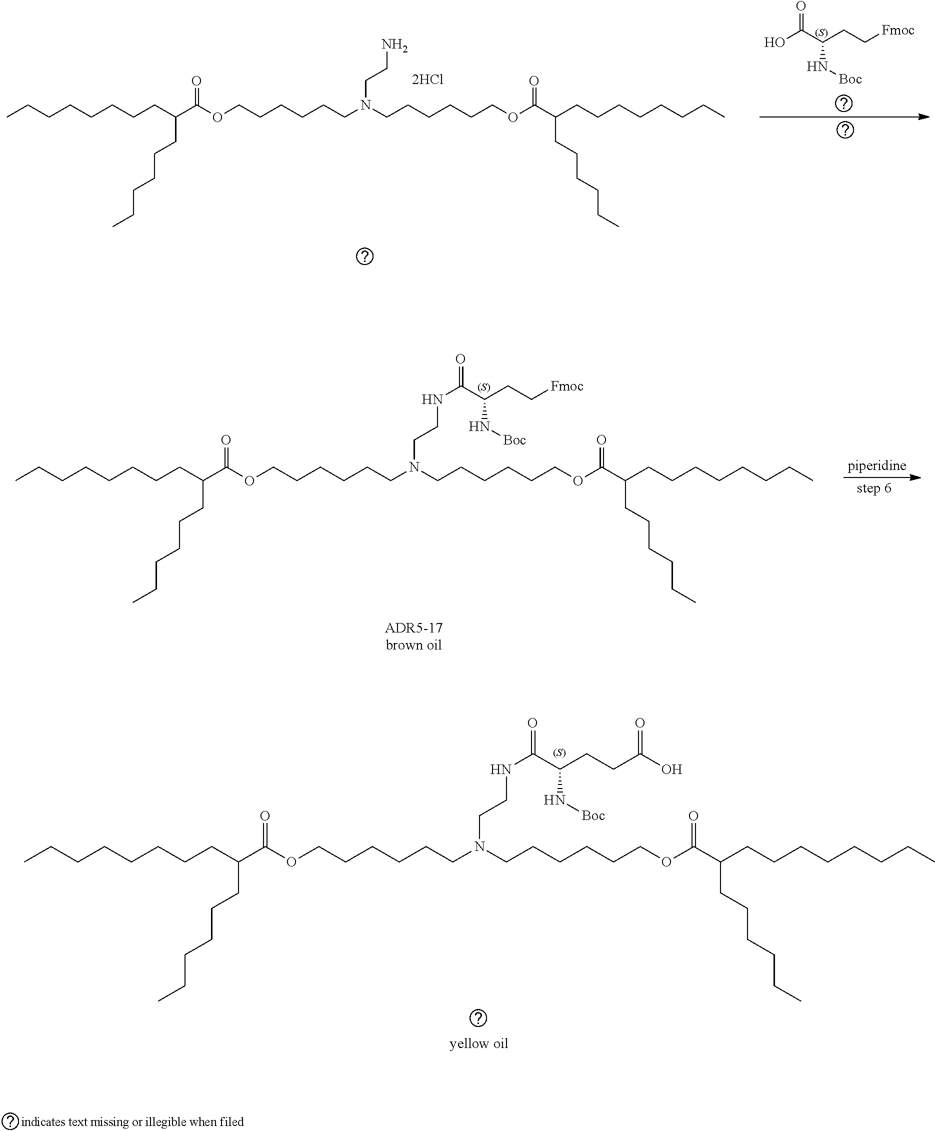

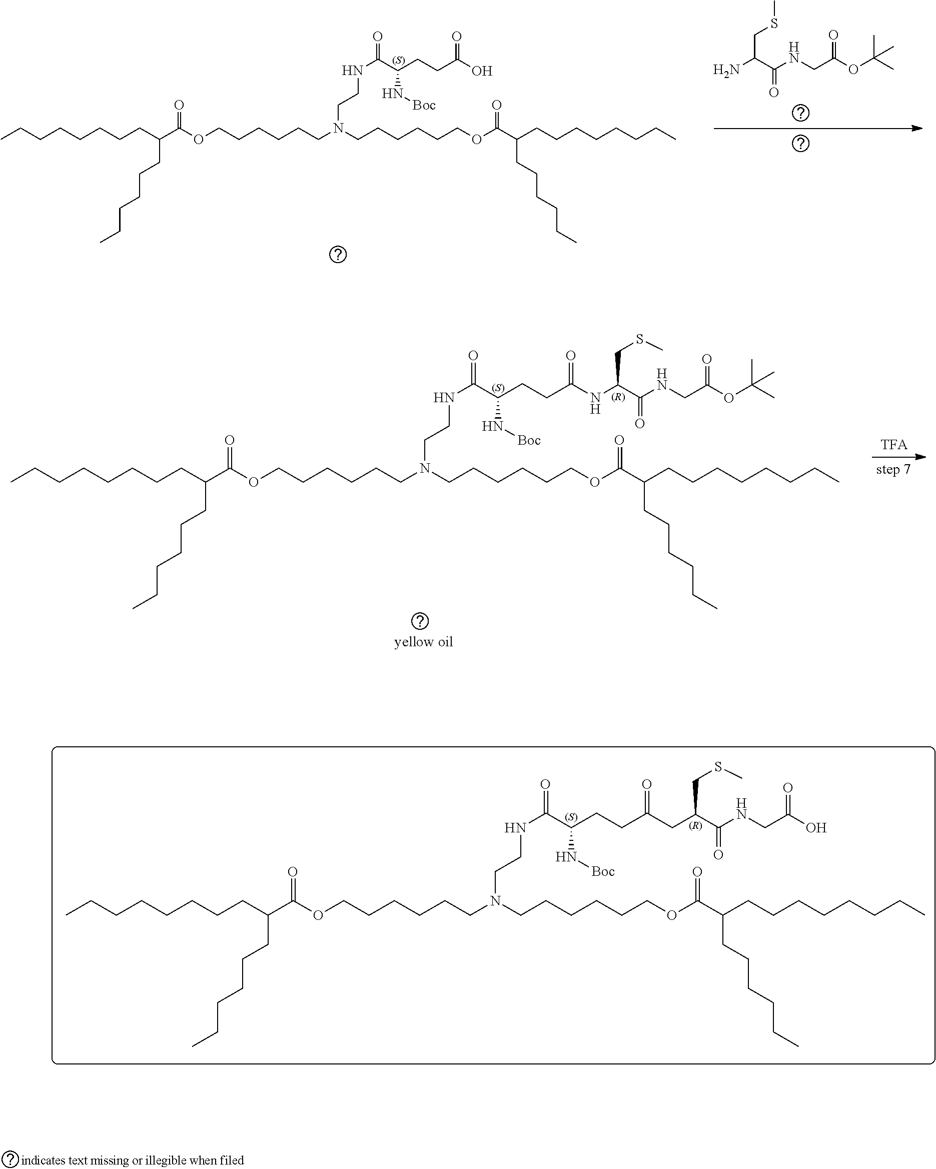

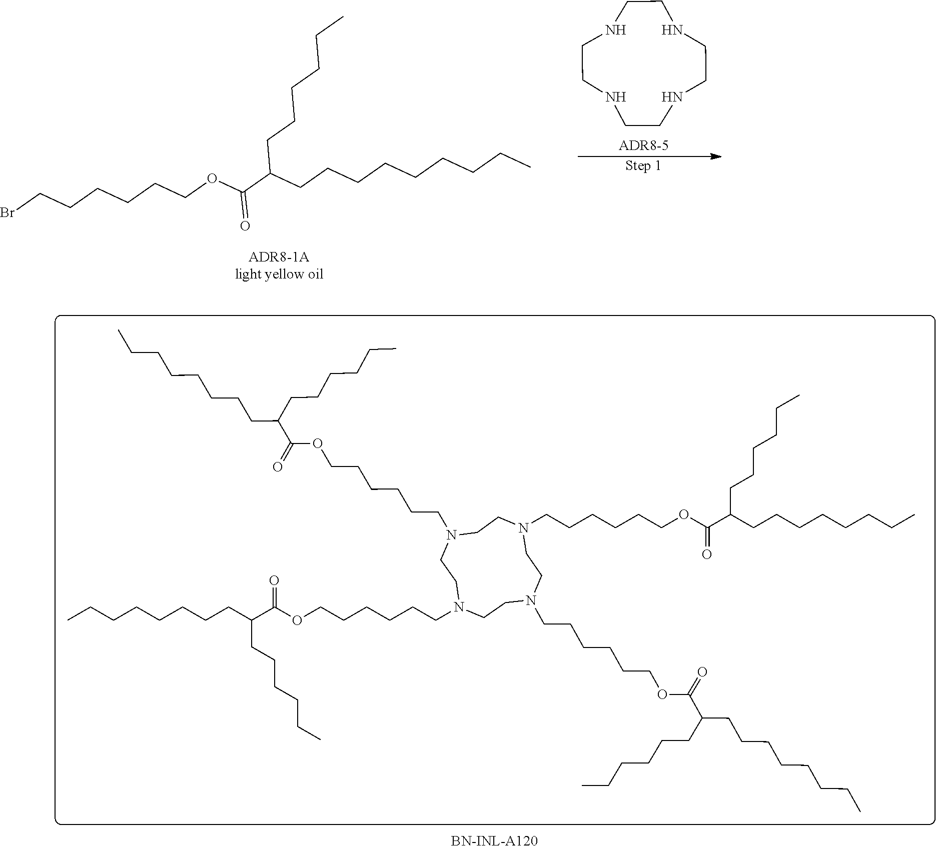 [SMILES: CCCCCCCCCC(CCCCCC)C(=O)OCCCCCCBr ; C1CNCCNCCNCCN1 ; "ADR8-1Alight yellow oil" ; CCCCCCCCC(CCCCCC)C(=O)OCCCCCCN1CCN(CCCCCCOC(=O)C(CCCCCC)CCCCCCCC)CCN(CCCCCCOC(=O)C(CCCCCC)CCCCCCCC)CCN(CCCCCCOC(=O)C(CCCCCC)CCCCCCCC)CC1]

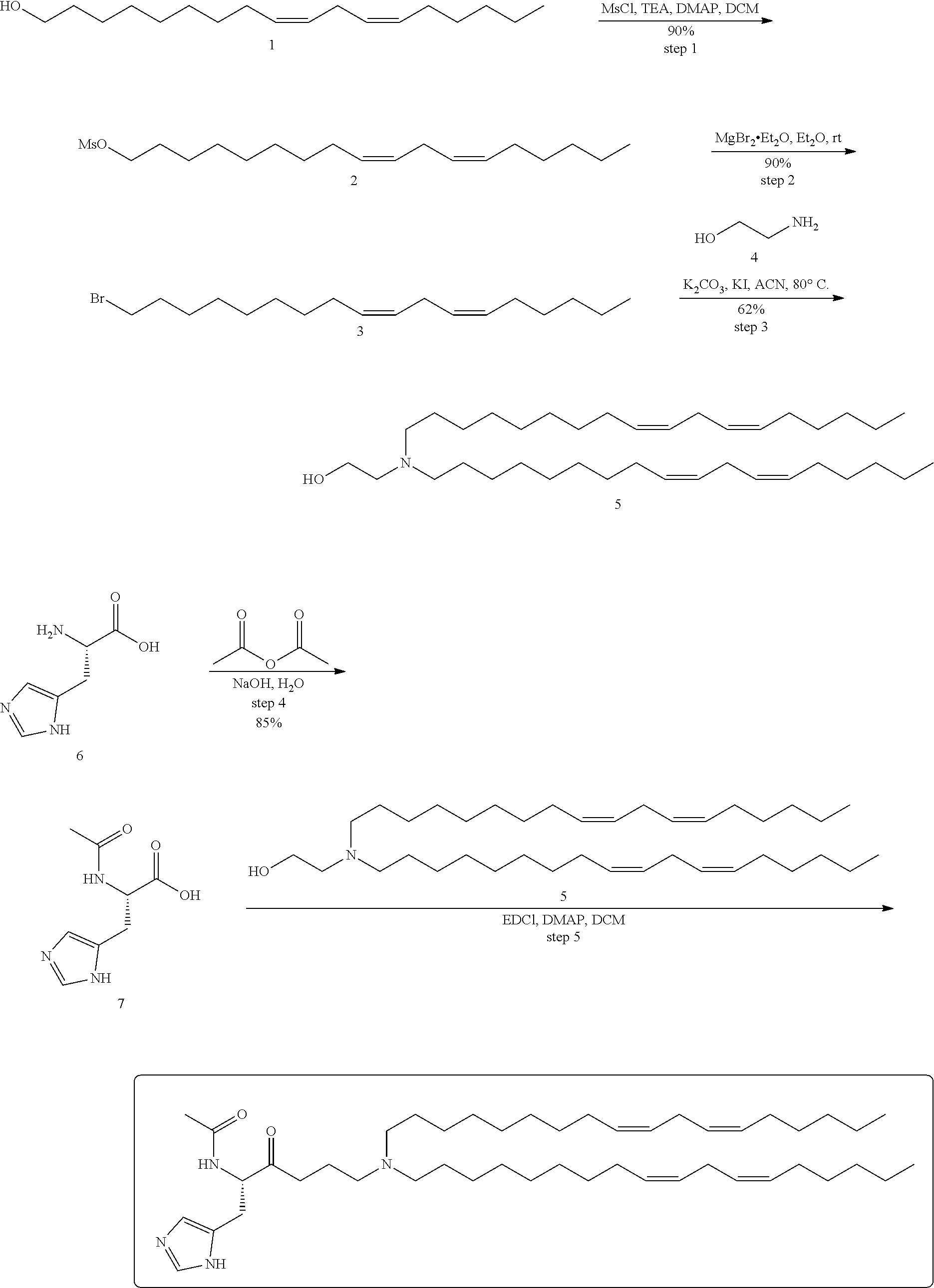

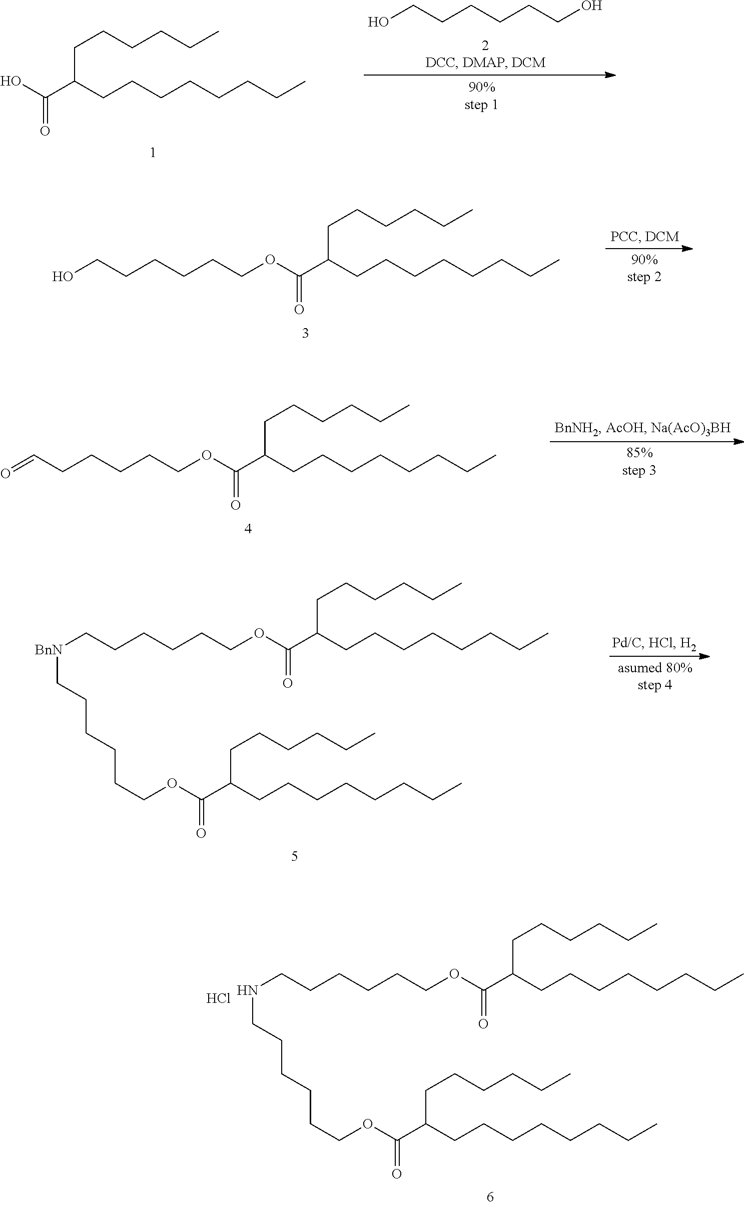

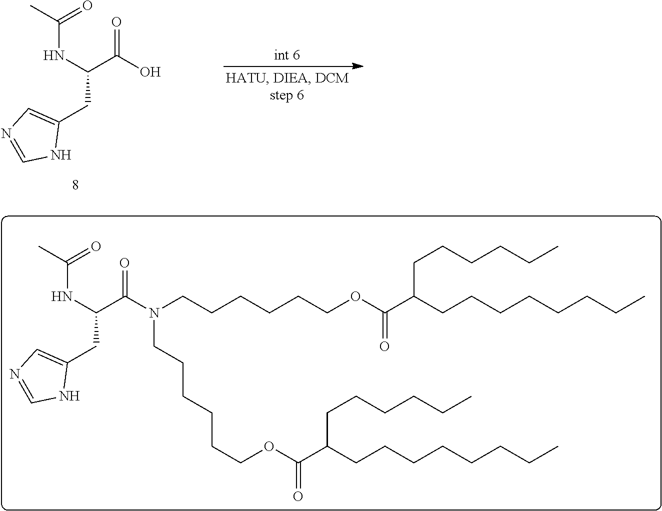 [SMILES: CC(=O)N[C@@H](Cc1cnc[nH]1)C(=O)O ; CCCCCCCCC(CCCCCC)C(=O)OCCCCCCN(CCCCCCOC(=O)C(CCCCCC)CCCCCCCC)C(=O)[C@H](Cc1cnc[nH]1)NC(C)=O]

Optional synthetic scheme for BN-INL-A106 is as follows:

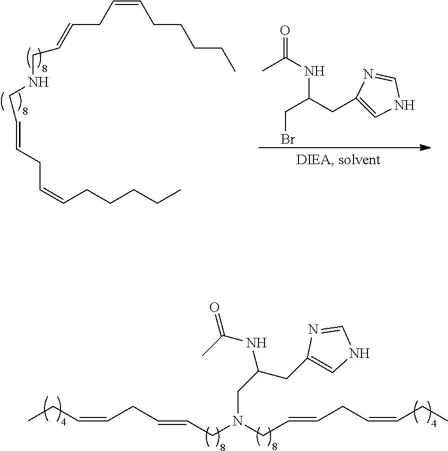

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

1. A compound represented by Formula 1: 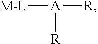
wherein:
M represents an ionizable moiety;
said ionizable moiety is selected from (i) an ionizable moiety comprising a heteroaryl; (ii) an ionizable moiety having a binding affinity to a CNS receptor; and
(iii) an ionizable moiety comprising a squaramide;
L represents a spacer or is a bond;
A represents a N, O, S, or C(H)₁₋₂ as allowed by valency;
each R independently represents H, a hydrocarbon comprising between 10 and 50 carbon atoms, or is absent, and at least one R is said hydrocarbon;
said hydrocarbon comprises a C20-C50 alkyl, a C20-C50 alkenyl, a C20-C50 alkyl or alkenyl comprising one or more heteroatoms, or wherein said hydrocarbon is represented by Formula I: 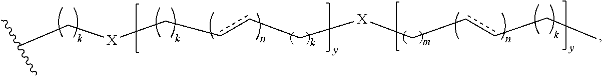
wherein: [symbol]
represents a single bond or a double bond;
k, and y, are integers each independently being between 1 and 10;
n and m are integers each independently being between 0 and 10;
each X independently comprises (i) CH₂, CHR₁, NR₁, NH, O, S, or phosphate; or
(ii) 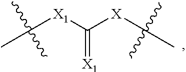
wherein each X₁ is independently O, NH, or S, and
wherein X is CH₂, CHR₁, NR₁, NH, O, or S;
and wherein R₁ comprises a C1-C20 alkyl, a C1-C20 alkenyl, a C20-C50 alkyl or alkenyl comprising one or more heteroatoms, or said hydrocarbon of Formula I; or
wherein the compound is represented by Formula 2: 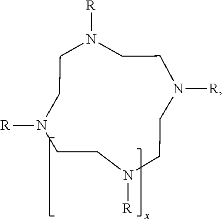
or by Formula 3: 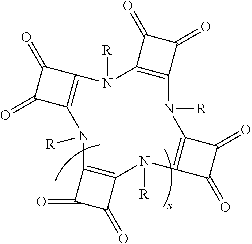
wherein:
x is between 1 and 4;
each R independently represents H, a hydrocarbon comprising between 10 and 50 carbon atoms, or is absent, and at least one R is said hydrocarbon;
said hydrocarbon comprises a C20-C50 alkyl, a C20-C50 alkenyl, a C20-C50 alkyl or alkenyl comprising one or more heteroatoms, or said hydrocarbon is represented by Formula I: 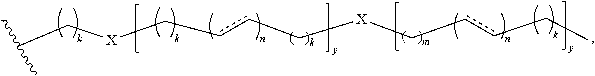
wherein: [symbol]
represents a single bond or a double bond;
k, and y, are integers each independently being between 1 and 10;
n and m are integers each independently being between 0 and 10;
each X independently comprises (i) CH2, CHR1, NR1, NH, O, S, or phosphate; or
(ii) 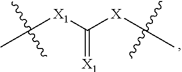
wherein each X1 is independently O, NH, or S, and
wherein X is CH2, CHR1, NR1, NH, O, or S;
and wherein R¹ comprises a C1-C20 alkyl, a C1-C20 alkenyl, a C20-C50 alkyl or alkenyl comprising one or more heteroatoms, or said hydrocarbon of Formula I.

2. The compound of claim 1, wherein said heteroaryl of the ionizable moiety is characterized by a pKa between 6 and 7.

3. The compound of claim 1, wherein said heteroaryl is imidazole.

4. The compound of claim 1, wherein the targeting moiety is selected from the group consisting of adenine, adenosine, glutathione, caffeine, or any combination thereof.

5. The compound of claim 1, wherein the ionizable moiety has a molecular weight (MW) of less than 1000 Da.

6. (canceled)

7. The compound of claim 1, wherein said compound is selected from: 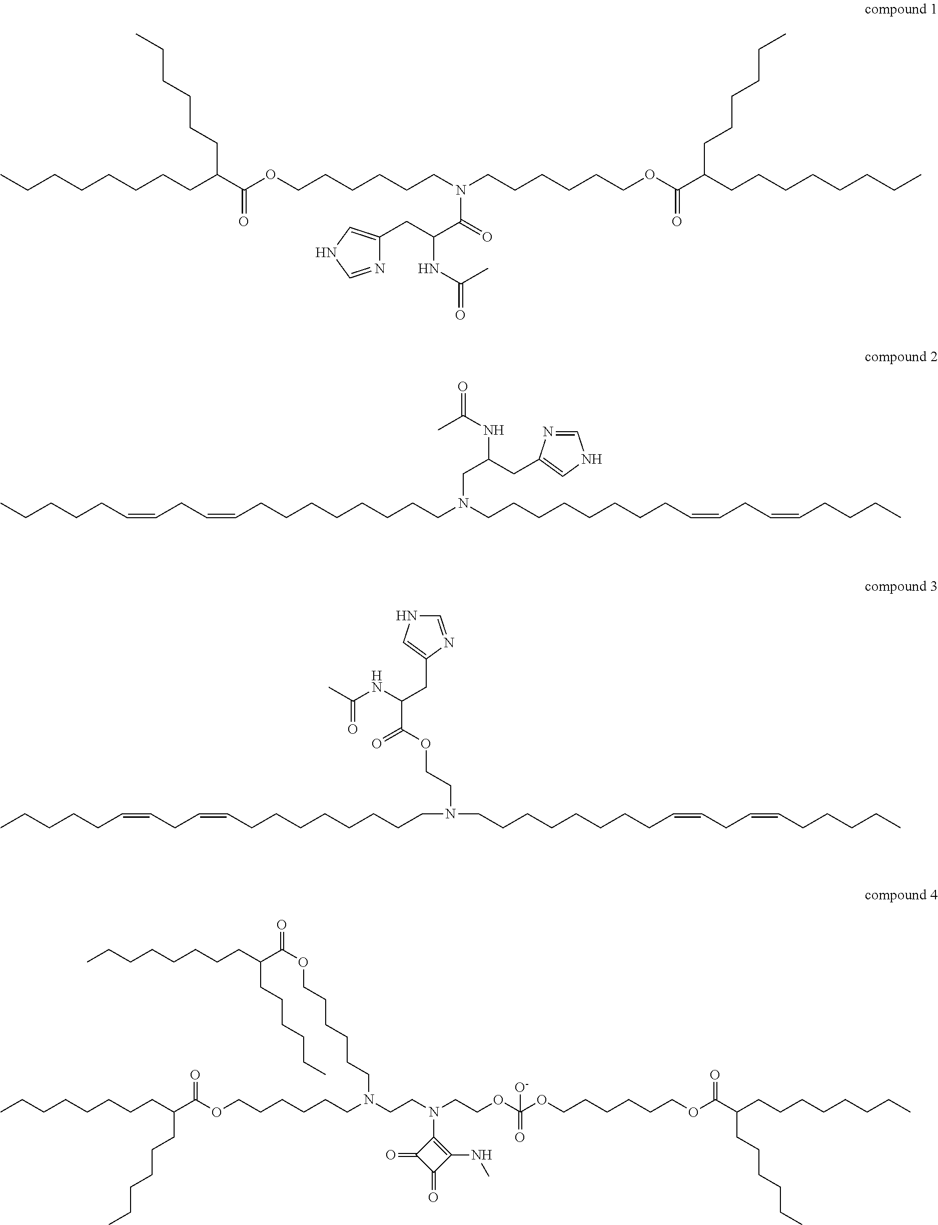 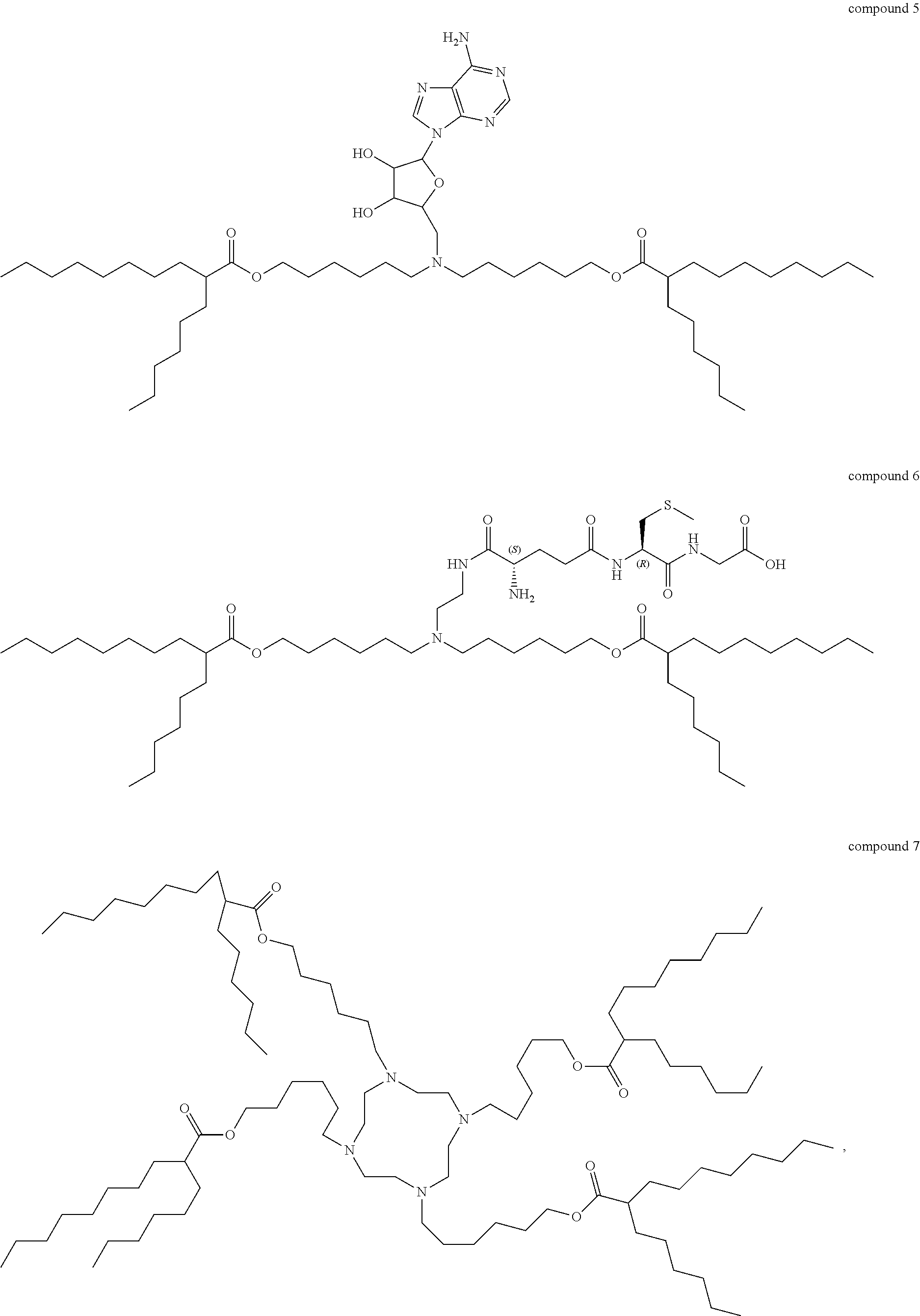
and wherein said compound includes any salt, any stereoisomer, or any tautomer thereof.

8. A nanoparticle comprising a core and a shell:
(i) the shell comprises a lipid, and at least one compound of claim 1;
(ii) the core is an aqueous core, comprising an active agent;
and wherein an average size of the nanoparticle is in a range between 30 and 300 nm.

9. The nanoparticle of claim 8, wherein a molar concentration of the compound within the nanoparticle is between 10 and 80 mol %; and wherein the shell further comprises a sterol; and wherein said sterol is cholesterol.

10. (canceled)

11. The nanoparticle of claim 2, wherein a molar concentration of the sterol within the nanoparticle is between 20 and 60 mol %; wherein a molar concentration of the lipid within the nanoparticle is between 3 and 40 mol %.

12. (canceled)

13. (canceled)

14. The nanoparticle of claim 8, wherein said lipid further comprises a PEG-lipid; and wherein a molar concentration of the PEG-lipid within the nanoparticle is between 0.1 and about 5 mol %; optionally wherein said lipid is a phospholipid.

15. The nanoparticle of claim 8, wherein said nanoparticle is a lipid nanoparticle (LNP); and optionally wherein said LNP is characterized by an average particle size between about 50 and about 250 nm, as determined by dynamic light scattering (DLS).

16. The nanoparticle of claim 15, wherein said LNP comprises a molar concentration of: (i) said compound between 30 and 60%, (ii) said lipid between 5 and 50%, (iii) said sterol between 25 and 50%, and (vi) said PEG-lipid between 0.5 and 5%; and wherein said active agent is a polynucleotide.

17. (canceled)

18. (canceled)

19. (canceled)

20. (canceled)

21. (canceled)

22. (canceled)

23. (canceled)

24. (canceled)

25. A pharmaceutical composition comprises a plurality of the nanoparticles of claim 8 and a pharmaceutically acceptable carrier.

26. The pharmaceutical composition of claim 25, comprising an effective amount of the active agent.

27. (canceled)

28. The pharmaceutical composition of claim 25, comprising a plurality of types of nanoparticles wherein said plurality comprises at least two nanoparticle types that differ in their lipid composition, their active agent, their nucleic acid molecule or a combination thereof, wherein said nucleic acid molecule uniquely identifies each nanoparticle type.

29. (canceled)

30. (canceled)

31. (canceled)

32. (canceled)

33. A method for delivering of an active agent to a specific tissue of a subject, comprising administering to the subject a therapeutically effective amount of the pharmaceutical composition of claim 25.

34. The method of claim 33, wherein said tissue is selected from tumor, brain tissue, lung tissue, spleen tissue, liver tissue, kidney tissue, and heart tissue; and wherein said active agent is a polynucleic acid.

35. (canceled)

36. (canceled)
